(19) United States
(12) Patent Application Publication
(10) Pub. No.: US 2011/0177997 A1
(43) Pub. Date: Jul. 21, 2011

(54) Cross-Beta Silk Genes

(76) Inventors: Tara D. Sutherland, Australian Capital Territory (AU); Victoria S. Haritos, Australian Capital Territory (AU); Alagacone Sriskantha, Australian Capital Territory (AU); Sarah Weisman, Australian Capital Territory (AU); Shoko Okada, Australian Capital Territory (AU); Holly Trueman, Australian Capital Territory (AU); Ajay Pundalikrao Niranjane, Australian Capital Territory (AU)

(21) Appl. No.: 12/672,487

(22) PCT Filed: Aug. 5, 2008
(86) PCT No.: PCT/AU08/01131

§ 371 Date: Apr. 4, 2011

Related U.S. Application Data
(60) Provisional application No. 60/954,189, filed on Aug. 6, 2007.

(51) Int. Cl. A61K 38/00; C12N 15/12; C12N 15/63; C12N 1/00; C07K 14/435; C12N 5/00; A01H 5/00; A01K 67/00; C12P 21/00; A61K 48/00; A61K 47/42

(52) U.S. Cl. 514/1.1; 536/23.5; 435/320.1; 435/243; 530/353; 435/325; 800/298; 800/13; 435/69.1; 514/44 R; 514/773

(57) ABSTRACT

The present invention relates to silk proteins which can be used to produce silk with a cross-beta structure, as well as nucleic acids encoding such proteins. The present invention also relates to recombinant cells and/or organisms which synthesize silk proteins. Silk proteins of the invention can be used for a variety of purposes such as in the production of personal care products, plastics, textiles, and biomedical products.

### FIELD OF THE INVENTION

The present invention relates to silk proteins which can be used to produce silk with a cross-beta structure, as well as nucleic acids encoding such proteins. The present invention also relates to recombinant cells and/or organisms which synthesize silk proteins. Silk proteins of the invention can be used for a variety of purposes such as in the production of personal care products, plastics, textiles, and biomedical products.

### BACKGROUND OF THE INVENTION

Silks are fibrous protein secretions that exhibit exceptional strength and toughness and as such have been the target of extensive study. Silks are produced by over 30,000 species of spiders and by many insects. Very few of these silks have been characterised, with most research concentrating on the cocoon silk of the domesticated silkworm, Bombyx mori and on the dragline silk of the orb-weaving spider Nephila clavipes.

In the Lepidoptera and spider, the fibroin silk genes code for proteins that are generally large with prominent hydrophilic terminal domains at either end spanning an extensive region of alternating hydrophobic and hydrophilic blocks (Bini et al., 2004). Generally these proteins comprise different combinations of crystalline arrays of β-pleated sheets loosely associated with β-sheets, β-spirals, α-helices and amorphous regions (see Craig and Riekel, 2002 for review).

As silk fibres represent some of the strongest natural fibres known, they have been subject to extensive research in attempts to reproduce their synthesis. However, a recurrent problem with expression of Lepidopteran and spider fibroin genes has been low expression rates in various recombinant expression systems due to the combination of the repeating nucleotide motifs in the silk gene that lead to deleterious recombination events, the large gene size and the small number of codons used for each amino acid in the gene which leads to depletion of tRNA pools in the host cells.

Recombinant expression leads to difficulties during translation such as translational pauses as a result of codon preferences and codon demands and extensive recombination rates leading to truncation of the genes. Shorter, less repetitive sequences would avoid many of the problems associated with silk gene expression to date.

In contrast to the extensive knowledge that has accumulated about the Lepidopteran (in particular the cocoon silk of Bombyx mori) and spider (in particular the dragline silk of Nephila clavipes) little is known about the chemical composition and molecular organisation of other insect silks. For example, less studied are silks based on other structures such as cross beta silks where the beta crystallites are orientated perpendicular to the silk fibre direction.

Silks with cross beta structure have been described in species of Neuroptera, Coleoptera, Hymenoptera and Diptera (Neuroptera: Chrysopa; Rudall and Kenchington, 1971; Coleoptera Hydrophilus; Rudall, 1962; Hypera sp; Kenchington 1983; Diptera: Arachnocampa luminosa; Rudall, 1962; Hymenoptera: Nematus ribessi).

As part of the egg laying process, the adult female lacewings spreads a film of silk from the malpigian tubules onto the substratum and then draws out a single fiber from the middle of this film (Rudall and Kenchington, 1971). The X-ray diffraction patterns and infra-red absorption spectra of both the film and fiber suggest they are comprised of proteins in a cross beta structure (Parker and Rudall, 1957; Rudall and Kenchington, 1971). The fibers can be extended to six times their initial length before breaking and in the stretched form have a parallel beta structure (Parker and Rudall, 1957).

Based on a detailed interpretation of the X-ray diffraction pattern (Parker and Rudall, 1957; Geddes et al., 1968), the observed extensibility of the silk (Parker and Rudall, 1957), the amino acid composition (Lucas et al., 1957) and skeletal models that were constructed to identify which amino acid residues would allow a protein backbone to form the turns in a cross beta sheet protein (Geddes et al., 1968), a model was proposed by Geddes et al. (1968) for the molecular structure of the egg-stalk silk from female adult C. flava. This model predicted flat protein ribbons of 25 Å in width, a finding later confirmed in preparations of silk gland proteins from female adult C. flava (Rudall and Kenchington, 1971).

Hepburn et al. (1979) investigated the stress-strain curve of the egg stalks of the green lacewing species Chrysopa carnea (Neuroptera:Chrysopidae). The tensile strength of the egg stalks is approximately 380 MPa. The tensile behavior of the silk during extension is consistent with unfolding from a cross-beta to a parallel beta conformation.

A slightly different cross beta structure has been described in several weevil species (Hypera sp., Coleoptera: Curculionidae—Kenchington, 1983). X-ray data and electron microscopy measurements of dispersed silk from the silk gland indicate that the larval cocoon silk of these species has a micellar width of 30 Å (rather than the 25 Å width of lacewing silk ribbons). This suggests a cross beta structure of greater than eight residues in length. A greater variety of amino acids make up this silk suggesting that it is chemically more complex than the Chrysopa flava silk.

Naturally occurring cross-beta proteins are rare. The main examples of evolved cross-beta fibers are in other arthropod silks, such as the capture threads of glow-worms (Rudall, 1962) and the cocoons of weevils (Kenchington, 1982), or in viral attachment fibers (Green et al., 1983). The apparent absence of cross-beta filaments in cellular environments may be due to their potential for extensive aggregation or nucleation of amyloid formation.

Hepburn et al. (1979) measured the tensile properties of the egg stalks of Chrysopa carnea, a second green lacewing species. The stalks were found to have both reasonable tensile strength (˜100 MPa) and very high extensibility (up to 600%). This combination of properties gives green lacewing egg stalk silk potential applications as a biomaterial.

Considering the unique properties of silks produced by insects, such as from Neuropterans, and that they are available naturally in only minute amounts, there is a need for the identification of further novel nucleic acids encoding silk proteins.

### SUMMARY OF THE INVENTION

The present inventors have identified numerous polynucleotides encoding silk proteins.

Thus, in a first aspect the present invention provides an isolated and/or exogenous polynucleotide which encodes a silk polypeptide, wherein at least a portion of silk comprising the polypeptide has a cross beta structure.

In one embodiment, the polynucleotide comprises:

i) a sequence of nucleotides as provided in any one of SEQ ID NO's 12 to 20;

ii) a sequence of nucleotides encoding a polypeptide comprising an amino acid sequence as provided in any one of SEQ ID NO's 1 to 9;

iii) a sequence of nucleotides encoding a polypeptide comprising an amino acid sequence which is at least 30% identical to any one or more of SEQ ID NO's 1 to 9;

iv) a sequence of nucleotides encoding a biologically active fragment of ii) or iii),

v) a sequence of nucleotides which is at least 30% identical to any one or more of SEQ ID NO's 12 to 20, and/or

vi) a sequence which hybridizes to any one of i) to v) under stringent conditions.

In another embodiment, the polynucleotide comprises:

i) a sequence of nucleotides as provided in SEQ ID NO:21 or SEQ ID NO:22;

ii) a sequence of nucleotides encoding a polypeptide comprising an amino acid sequence as provided in SEQ ID NO:10 or SEQ ID NO:11;

iii) a sequence of nucleotides encoding a polypeptide comprising an amino acid sequence which is at least 30% identical to SEQ ID NO:10 and/or SEQ ID NO:11;

iv) a sequence of nucleotides encoding a biologically active fragment of ii) or iii),

v) a sequence of nucleotides which is at least 30% identical to SEQ ID NO:21 and/or SEQ ID NO:22, and/or

vi) a sequence which hybridizes to any one of i) to v) under stringent conditions.

In a particularly preferred embodiment, the polynucleotide encodes a polypeptide of the invention.

In another aspect, the present invention provides a vector comprising at least one polynucleotide of the invention.

In a preferred embodiment, the vector is an expression vector. More preferably, the polynucleotide is operably linked to a promoter in the expression vector.

In a further aspect, the present invention provides a host cell comprising at least one polynucleotide of the invention, and/or at least one vector of the invention.

The host cell can be any cell type. Examples include, but are not limited to, a bacterial, yeast or plant cell.

In a further aspect, the present invention provides a substantially purified and/or recombinant silk polypeptide, wherein at least a portion of silk comprising the polypeptide has a cross beta structure.

In an embodiment, the polypeptide comprises at least 30% serine, at least 15% glycine and at least 15% alanine.

In another embodiment, the polypeptide comprises a beta sheet comprising at least 50 strands, wherein each strand is 8 amino acids in length.

In a further embodiment, the polypeptide comprises:

i) an amino acid sequence as provided in any one of SEQ ID NO's 1 to 9;

ii) an amino acid sequence which is at least 30% identical to any one or more of SEQ ID NO's 1 to 9; and/or

iii) a biologically active fragment of i) or ii).

More preferably, the polypeptide of this embodiment comprises between 38% and 48% serine, between 22% and 32% glycine, and between 14% and 24% alanine. Even more preferably, the polypeptide of this embodiment comprises between 41% and 45% serine, between 25% and 29% glycine, and between 17% and 21% alanine.

In a further aspect, the polypeptide comprises:

i) an amino acid sequence as provided in SEQ ID NO:10 or SEQ ID NO:11;

ii) an amino acid sequence which is at least 30% identical to SEQ ID NO:10 and/or SEQ ID NO:11; and/or

iii) a biologically active fragment of i) or ii).

More preferably, the polypeptide of this embodiment comprises between 29% and 39% serine, between 16% and 26% glycine, and between 21% and 31% alanine.

Even more preferably, the polypeptide of this embodiment comprises between 32% and 36% serine, between 19% and 23% glycine, and between 24% and 28% alanine.

Preferably, the polypeptide can be purified from a species of Neuroptera, Diptera, Hymenoptera or Coleoptera. More preferably, the polypeptide can be purified from a species of Neuroptera.

Preferably, the species of Neuroptera is Mallada signata.

In a further embodiment, the polypeptide is fused to at least one other polypeptide. In a preferred embodiment, the at least one other polypeptide is selected from the group consisting of: a polypeptide that enhances the stability of a polypeptide of the present invention, a polypeptide that assists in the purification of the fusion protein, and a polypeptide which assists in the polypeptide of the invention being secreted from a cell (for example secreted from a plant cell).

In yet another aspect, the present invention provides a transgenic plant comprising an exogenous polynucleotide of the invention, the polynucleotide encoding at least one polypeptide according of the invention.

In another aspect, the present invention provides a transgenic non-human animal comprising an exogenous polynucleotide of the invention, the polynucleotide encoding at least one polypeptide of the invention.

Also provided is a process for preparing a polypeptide of the invention, the process comprising cultivating a host cell of the invention, a vector of the invention, a plant of the invention or a non-human animal of the invention, under conditions which allow expression of the polynucleotide encoding the polypeptide, and recovering the expressed polypeptide.

In a further aspect, the present invention provides an isolated and/or recombinant antibody which specifically binds a polypeptide of the invention.

In another aspect, the present invention provides a silk fiber comprising at least one polypeptide of the invention.

Preferably, the polypeptide is a recombinant polypeptide.

In a further aspect, the present invention provides a copolymer comprising at least two polypeptides of the invention.

Preferably, the polypeptides are recombinant polypeptides.

Preferably, the copolymer comprises polypeptides comprising:

i) an amino acid sequence as provided in any one of SEQ ID NO's 1 to 9;

ii) an amino acid sequence which is at least 30% identical to any one or more of SEQ ID NO's 1 to 9; and/or

iii) a biologically active fragment of i) or ii),

and polypeptides comprising:

i) an amino acid sequence as provided in SEQ ID NO:10 or SEQ ID NO:11;

ii) an amino acid sequence which is at least 30% identical to SEQ ID NO:10 and/or SEQ ID NO:11; and/or

iii) a biologically active fragment of i) or ii),

in a molar ratio of about 7:1.

In an alternate embodiment, the more ratio is not about 7:1.

In another aspect, the present invention provides a product comprising at least one polypeptide of the invention, at least one silk fiber of the invention and/or at least one copolymer of the invention.

Examples of products of the invention include, but are not limited to, a personal care product, textiles, plastics, and biomedical products.

In a further aspect, the present invention provides a composition comprising at least one polypeptide of the invention, at least one silk fiber of the invention and/or at least one copolymer of the invention, and one or more acceptable carriers.

In an embodiment, the composition further comprises a drug.

In another embodiment, the composition is for use as a medicine, a medical device or a cosmetic.

In yet another aspect, the present invention provides a composition comprising at least one polynucleotide of the invention, and one or more acceptable carriers.

In a further aspect, the present invention provides a method of treating or preventing a disease, the method comprising administering a composition comprising at least one drug for treating or preventing the disease and a pharmaceutically acceptable carrier, wherein the pharmaceutically acceptable carrier is selected from at least one polypeptide of the invention, at least one silk fiber of the invention, at least one copolymer of the invention, at least one product of the invention and/or at least one composition of the invention.

Also provided is the use of at least one polypeptide of the invention, at least one silk fiber of the invention, at least one copolymer of the invention, at least one product of the invention and/or at least one composition of the invention, and at least one drug, for the manufacture of a medicament for treating or preventing a disease.

Furthermore, provided is the use of at least one polypeptide of the invention, at least one silk fiber of the invention, at least one copolymer of the invention, at least one product of the invention and/or at least one composition of the invention, and at least one drug, as a medicament for treating or preventing a disease.

In yet another aspect, the present invention provides a kit comprising at least one polypeptide of the invention, at least one polynucleotide of the invention, at least one vector of the invention, at least one silk fiber of the invention, at least one copolymer of the invention, at least one product of the invention and/or at least one composition of the invention.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. The predicted amino acid composition of the silk proteins from Mallada signata (Chrysopidae) (MalXBFibroin and MalXBsFib in 7:1 molar ratio) matches the amino acid composition measured in the native silk of Chrysopa flava by Lucas et al. (1957).

FIG. 2. Sequence and architecture of MalXBFibroin (SEQ ID NO:1) and of MalXBsFib (SEQ ID NO:10). Both proteins start with a signal peptide. Grey shading indicates conserved amino acids in repetitive regions with 16-residue periodicity. Cysteine residues are framed.

FIG. 3. Synchrotron wide-angle x-ray scattering from Mallada signata egg stalks. A: scattering pattern with fiber axis vertical; the central ring is an artefact due to a kapton membrane. B: diagram of proposed quarter-staggered cross-beta protein structure with assignment of axes. C: cross-section of pseudo-cell in be plane; up or down pointing triangles indicate amino acids with side chains projecting up or down. D: cross-section of pseudo-cell in ac plane; black or white circles indicate protein chains running into or out of the page. E: table of scattering peaks and assignments comparing measured positions to positions calculated from pseudo-cell dimensions.

FIG. 4. Proposed cross-beta sequence-structure model for Mallada signata egg stalk silk proteins. A: first repetitive region of MalXBFibroin (SEQ ID NO:23), B: second repetitive region of MalXBFibroin (SEQ ID NO:24), C: repetitive region of MalXBsFib (SEQ ID NO:25). Bold letters indicate residues more bulky than serine; blue letters indicate charged or highly polar residues. Dark red Roman numerals adjacent to turns indicate the type of beta-turn as predicted by the COUDES algorithm (Fuchs and Alix, 2005).

FIG. 5. DNA dot plots showing that MalXBFibroin (A) is significantly less repetitive than a partial sequence of silkworm heavy fibroin gene (B).

FIG. 6. Codon usage graph showing less bias for MalXBFibroin than for the silkworm heavy fibroin gene.

FIG. 7. The different MalXBFibroin cDNA contain many insertions and/or deletions and 84 single nucleotide polymorphisms relative to each other but encode only seven non-synonymous changes. The cDNA were manually aligned to minimise the number of single nucleotide polymorphisms between sequences. A: Sequence coverage of the six (1-7) isolated silk gene cDNA. B: Comparison of the structure of the different silk gene cDNAs showing insertions (above the line) and deletions (below the line). Numbers in bracket indicate which cDNA the insertions or deletions correspond too. C: Position of single nucleotide polymorphisms with amino acid change indicated for the seven non-synonymous changes. The dotted line indicates the region encoding the cross beta sheet sequence shown in FIG. 4.

FIG. 8. Expression of lacewing cross beta protein. M: marker; 1: unpurified protein; 2: purified protein showing band at expected size (80 KDa).

### KEY TO THE SEQUENCE LISTING

SEQ ID NO:1—MalXBFibrion1 polypeptide sequence.

SEQ ID NO:2—MalXBFibrion1 polypeptide sequence without signal sequence.

SEQ ID NO:3—MalXBFibrion2 polypeptide sequence.

SEQ ID NO:4—MalXBFibrion2 polypeptide sequence without signal sequence.

SEQ ID NO:5—MalXBFibrion3 partial polypeptide sequence.

SEQ ID NO:6—MalXBFibrion4 partial polypeptide sequence. SEQ ID NO:7—MalXBFibrion5 partial polypeptide sequence.

SEQ ID NO:8—MalXBFibrion6 partial polypeptide sequence.

SEQ ID NO:9—MalXBFibrion7 partial polypeptide sequence.

SEQ ID NO:10—MalXBsFib polypeptide sequence.

SEQ ID NO:11—MalXBsFib polypeptide sequence without signal sequence.

SEQ ID NO:12—Polynucleotide sequence encoding MalXBFibrion1.

SEQ ID NO:13—Polynucleotide sequence encoding MalXBFibrion1 without signal sequence.

SEQ ID NO:14—Polynucleotide sequence encoding MalXBFibrion2.

SEQ ID NO:15—Polynucleotide sequence encoding MalXBFibrion2 without signal sequence.

SEQ ID NO:16—Partial polynucleotide sequence encoding MalXBFibrion3.

SEQ ID NO:17—Partial polynucleotide sequence encoding MalXBFibrion4.

SEQ ID NO:18—Partial polynucleotide sequence encoding MalXBFibrion5.

SEQ ID NO:19—Partial polynucleotide sequence encoding MalXBFibrion6.

SEQ ID NO:20—Partial polynucleotide sequence encoding MalXBFibrion7.

SEQ ID NO:21—Polynucleotide sequence encoding MalXBsFib.

SEQ ID NO:22—Polynucleotide sequence encoding MalXBsFib without signal sequence.

SEQ ID NO:23—First repetitive region of MalXBFibroin.

SEQ ID NO:24—Second repetitive region of MalXBFibroin.

SEQ ID NO:25—Repetitive region of MalXBsFib.

SEQ ID NO's 26 to 31—Oligonucleotide primers.

SEQ ID NO:32—First repeat consensus of MalXBFibrion1.

SEQ ID NO:33—Second repeat consensus of MalXBFibrion1.

SEQ ID NO:34—Repeat consensus of MalXBFib.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, recombinant biology, silk technology, immunology, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used herein, the terms “silk protein” and “silk polypeptide” refer to a fibrous protein/polypeptide that can be used to produce a silk fibre, and/or a fibrous protein complex.

As used herein, the term “cross beta structure” refers to a polypeptide comprising regular beta turns that allow the protein backbone to fold back on itself to form beta sheets of regular length. In a preferred embodiment, the cross beta structure has stacked beta-strands running perpendicular to the direction of a silk fibre.

As used herein, the term “beta sheet” refers to the secondary structure of a proteins consisting of beta strands connected laterally by three or more hydrogen bonds, forming a generally twisted, pleated sheet.

As used herein, the phrase “at least a portion of silk comprising the polypeptide has a cross beta structure” refers to at least 50%, more preferably at least 60%, of the protein, when present in a silk fibre, having a cross beta structure. In a preferred embodiment, about 65% to about 78%, more preferably about 68% to 75%, of the protein, when present in a silk fibre, has a cross beta structure.

As used herein, a “silk fibre” refers to filaments comprising proteins of the invention which can be woven into various items such as textiles.

As used herein, a “copolymer” is composition comprising two or more silk proteins of the invention. This term excludes naturally occurring copolymers such as the egg stalk or brood comb of insects.

The term “plant” includes whole plants, vegetative structures (for example, leaves, stems), roots, floral organs/structures, seed (including embryo, endosperm, and seed coat), plant tissue (for example, vascular tissue, ground tissue, and the like), cells and progeny of the same.

A “transgenic plant” refers to a plant that contains a gene construct (“transgene”) not found in a wild-type plant of the same species, variety or cultivar. A “transgene” as referred to herein has the normal meaning in the art of biotechnology and includes a genetic sequence which has been produced or altered by recombinant DNA or RNA technology and which has been introduced into the plant cell. The transgene may include genetic sequences derived from a plant cell. Typically, the transgene has been introduced into the plant by human manipulation such as, for example, by transformation but any method can be used as one of skill in the art recognizes.

“Polynucleotide” refers to an oligonucleotide, nucleic acid molecule or any fragment thereof. It may be DNA or RNA of genomic or synthetic origin, double-stranded or single-stranded, and combined with carbohydrate, lipids, protein, or other materials to perform a particular activity defined herein.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

The term “signal peptide” refers to an amino terminal polypeptide preceding a secreted mature protein. The signal peptide is cleaved from and is therefore not present in the mature protein. Signal peptides have the function of directing and trans-locating secreted proteins across cell membranes. The signal peptide is also referred to as signal sequence.

As used herein, “transformation” is the acquisition of new genes in a cell by the incorporation of a polynucleotide.

As used herein, the term “drug” refers to any compound that can be used to treat or prevent a particular disease, examples of drugs which can be formulated with a silk protein of the invention include, but are not limited to, proteins, nucleic acids, anti-tumor agents, analgesics, antibiotics, anti-inflammatory compounds (both steroidal and non-steroidal), hormones, vaccines, labeled substances, and the like.

As used herein, unless stated to the contrary the phrase “about” refers to any reasonable range in light of the value in question. In a preferred embodiment, the term “about” refers to +/−10%, more preferably +/−5%, of the specified value.

### Polypeptides

By “substantially purified polypeptide” or “purified polypeptide” we mean a polypeptide that has generally been separated from the lipids, nucleic acids, other polypeptides, and other contaminating molecules such as wax with which it is associated in its native state. With the exception of other proteins of the invention, it is preferred that the substantially purified polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered, preferably increased, amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably and refer to a single polypeptide chain which may or may not be modified by addition of non-amino acid groups. The terms “proteins” and “polypeptides” as used herein also include variants, mutants, modifications, analogous and/or derivatives of the polypeptides of the invention as described herein.

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

As used herein a “biologically active” fragment is a portion of a polypeptide of the invention which maintains a defined activity of the full-length polypeptide, namely the ability to be used to produce silk. Biologically active fragments can be any size as long as they maintain the defined activity. Preferably, biologically active fragments are, where relevant, at least 300, more preferably at least 500, more preferably at least 600, and even more preferably at least 900 amino acids in length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). These DNA shuffling techniques may include genes of the invention possibly in addition to genes related to those of the present invention, such as silk genes from Neuroptean, Dipteran, Hymenopteran or Coleopteran species other than the specific species characterized herein. Products derived from mutated/altered DNA can readily be screened using techniques described herein to determine if they can be used as silk proteins.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to about 150 residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1
Exemplary substitutions

| Original | Exemplary |
|---|---|
| Residue | Substitutions |
| Ala (A) | val; leu; ile; gly; cys; ser; thr |
| Arg (R) | lys |
| Asn (N) | gln; his |
| Asp (D) | glu |
| Cys (C) | ser; thr; ala; gly; val |
| Gln (Q) | asn; his |
| Glu (E) | asp |
| Gly (G) | pro; ala; ser; val; thr |
| His (H) | asn; gln |
| Ile (I) | leu; val; ala; met |
| Leu (L) | ile; val; met; ala; phe |
| Lys (K) | arg |
| Met (M) | leu; phe |
| Phe (F) | leu; val; ala |
| Pro (P) | gly |
| Ser (S) | thr; ala; gly; val; gln |
| Thr (T) | ser; gln; ala |
| Trp (W) | tyr |
| Tyr (Y) | trp; phe |
| Val (V) | ile; leu; met; phe; ala; ser; thr |

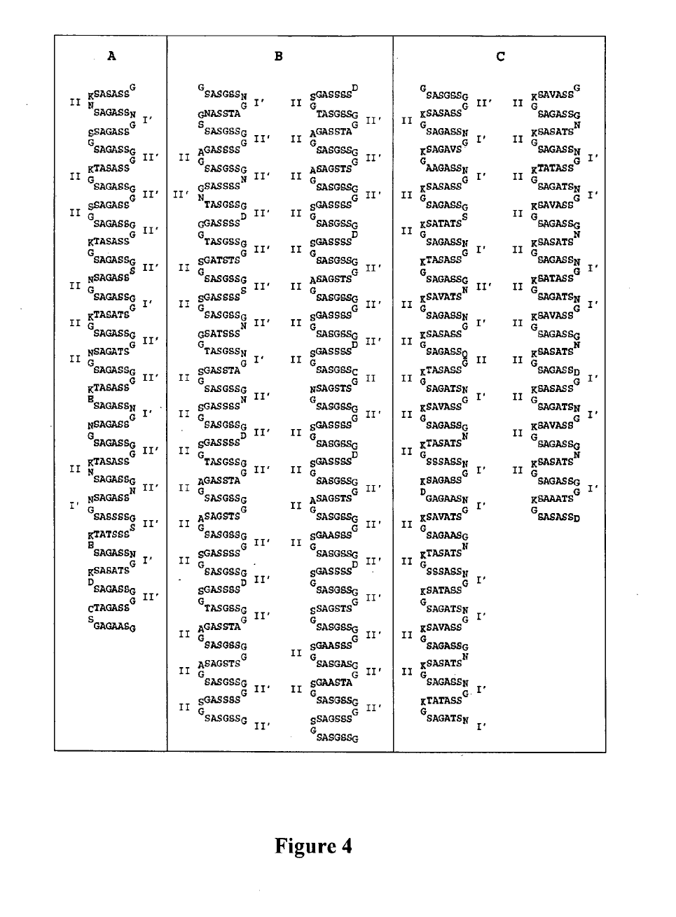

Preferably, the polypeptides of the invention comprise at least 50 strands, at least 60 strands, at least 70 strands, at least 80 strands, at least 90 strands, at least 100 strands or at least 110 strands. Preferably, each strand (beta sheet alternating with beta turns) is 6 to 10 amino acids in length, more preferably 8 amino acids in length. Examples of strands of 8 amino acids in length are provided in FIG. 4.

Preferably, at least 90% of the amino acids at position i of the beta turn on the right hand side are serine. More preferably, at least 95% of the amino acids at position i of the beta turn on the right hand side are serine. Even more preferably, 100% of the amino acids at position i of the beta turn on the right hand side are serine.

Four amino acids contribute to each beta turn (two residues from the top strand and two residues from the bottom strand of the beta sheet). These are conventionally described as ‘i’, ‘i+1’, ‘i+2’, ‘i’+3 where i is the first residue in the polypeptide chain that contributes to the turn, i+1 is the next residue and so on.

As used herein, right and left correspond to relative positions of the turn in FIG. 4.

When the polypeptide is closely related to any one of SEQ ID NO's 1 to 9 it is preferred that at least 40% of the amino acids at position i of the beta turn on the left hand side are glycine. Preferably, if glycine is not present the amino acid is serine, threonine or asparagine.

When the polypeptide is closely related to any one of SEQ ID NO's 1 to 9 it is preferred that at least 80% of the amino acids at position i+1 of the beta turn on the right hand side are glycine. Preferably, if glycine is not present the amino acid is cysteine, glutamine or asparagine.

When the polypeptide is closely related to any one of SEQ ID NO's 1 to 9 it is preferred that at least 35% of the amino acids at position i+1 of the beta turn on the left hand side are serine. Preferably, if serine is not present the amino acid is alanine, lysine, cysteine, glycine, or asparagine.

When the polypeptide is closely related to any one of SEQ ID NO's 1 to 9 it is preferred that at least 65% of the amino acids at position i+2 of the beta turn on the right hand side are glycine. Preferably, if glycine is not present the amino acid is serine, aspartic acid or asparagine.

When the polypeptide is closely related to any one of SEQ ID NO's 1 to 9 it is preferred that at least 75% of the amino acids at position i+2 of the beta turn on the left hand side are glycine. Preferably, if glycine is not present the amino acid is serine, aspartic acid, glutamic acid or asparagine.

When the polypeptide is closely related to any one of SEQ ID NO's 1 to 9 it is preferred that at least 80% of the amino acids at position i+3 of the beta turn on the right hand side are serine. Preferably, if serine is not present the amino acid is alanine.

When the polypeptide is closely related to any one of SEQ ID NO's 1 to 9 it is preferred that at least 80% of the amino acids at position i+3 of the beta turn on the left hand side are serine. Preferably, if serine is not present the amino acid is threonine, glycine or valine.

When the polypeptide is closely related to any one of SEQ ID NO's 1 to 9 it is preferred that at least 85% of the amino acids at position 1 of the internal beta sheet are alanine. Preferably, if alanine is not present the amino acid is serine, glycine, valine, cysteine or isoleucine.

When the polypeptide is closely related to any one of SEQ ID NO's 1 to 9 it is preferred that at least 60% of the amino acids at position 2 of the internal beta sheet are serine. Preferably, if serine is not present the amino acid is glycine, alanine, threonine or asparagine.

When the polypeptide is closely related to any one of SEQ ID NO's 1 to 9 it is preferred that at least 90% of the amino acids at position 3 of the internal beta sheet are serine, glycine and/or alanine, preferably an approximate equal amount of each. Preferably, if serine, glycine or alanine are not present the amino acid is valine or threonine.

When the polypeptide is closely related to any one of SEQ ID NO's 1 to 9 it is preferred that at least 70% of the amino acids at position 4 of the internal beta sheet are serine. Preferably, if serine is not present the amino acid is glycine, alanine, threonine or valine.

When the polypeptide is closely related to SEQ ID NO:1 or SEQ ID NO:2 it is preferred that the polypeptide comprises two regions of a 16 amino acid contiguous repeat. Preferably, the first region is at least 8 repeats, more preferably at least 12 repeats. In an alternate embodiment, the first region is about 15 repeats. Preferably, the second region is at least 25 repeats, more preferably at least 30 repeats. In an alternate embodiment, the second region is about 35 repeats. Preferably, the repeats of the first region comprise the sequence:

| | |
|---|---|
| X₁SX₂X₃X₄AX₂X₅X₆X₇AX₇X₃X₃SX₈. | (SEQ ID NO: 32) |

Preferably, the repeats of the second region comprise the sequence:

| | |
|---|---|
| X₉X₃X₂SX₁₀AX₁X₁₁X₁X₂ASGX₃SX₁₂. | (SEQ ID NO:33) |

### Where,

### X₁=Gly, Ser or Asn,

### X₂=Ser or Thr

### X₃=Ala or Ser

### X₄=Ser, Thr or Gly

### X₅=Lys, Ser, Asn, Cys

### X₆=Asn, Gly, Glu, Asp or Ser

### X₇=Ser or Gly

### X₈=Asn or Gly

### X₉=Gly, Ser, Asn or Asp,

### X₁₀=Gly, Thr, Ser or Ala,

### X₁₁=Ser, Asn, Gly or Ala, and

### X₁₂=Asn, Gly or Cys.

When the polypeptide is closely related to SEQ ID NO:11 or SEQ ID NO:12 it is preferred that at least 70% of the amino acids at position i of the beta turn on the left hand side are serine. Preferably, if serine is not present the amino acid is threonine.

When the polypeptide is closely related to SEQ ID NO:11 or SEQ ID NO:12 it is preferred that at least 80% of the amino acids at position i+1 of the beta turn on the right hand side are asparagine and/or glycine. Preferably, if asparagine or glycine are not present the amino acid is aspartic acid or glutamic acid.

When the polypeptide is closely related to SEQ ID NO:11 or SEQ ID NO:12 it is preferred that at least 90% of the amino acids at position i+1 of the beta turn on the left hand side are lysine. Preferably, if lysine is not present the amino acid is glycine. When the polypeptide is closely related to SEQ ID NO:11 or SEQ ID NO:12 it is preferred that at least 90% of the amino acids at position i+2 of the beta turn on the right hand side are asparagine and/or glycine. Preferably, if asparagine or glycine are not present the amino acid is serine.

When the polypeptide is closely related to SEQ ID NO:11 or SEQ ID NO:12 it is preferred that at least 90% of the amino acids at position i+2 of the beta turn on the left hand side are glycine. Preferably, if lysine is not present the amino acid is aspartic acid.

When the polypeptide is closely related to SEQ ID NO:11 or SEQ ID NO:12 it is preferred that at least 95% of the amino acids at position i+3 of the beta turn on the right hand side are serine. Preferably, all of the amino acids are serine.

When the polypeptide is closely related to SEQ ID NO:11 or SEQ ID NO:12 it is preferred that at least 85% of the amino acids at position i+3 of the beta turn on the left hand side are serine. Preferably, if serine is not present the amino acid is alanine or glycine.

When the polypeptide is closely related to SEQ ID NO:11 or SEQ ID NO:12 it is preferred that at least 90% of the amino acids at position 1 of the internal beta sheet are alanine. Preferably, if alanine is not present the amino acid is serine or glycine.

When the polypeptide is closely related to SEQ ID NO:11 or SEQ ID NO:12 it is preferred that at least 60% of the amino acids at position 2 of the internal beta sheet are glycine and/or serine. Preferably, if glycine and/or serine are not present the amino acid is valine, threonine, alanine or cysteine.

When the polypeptide is closely related to SEQ ID NO:11 or SEQ ID NO:12 it is preferred that at least 90% of the amino acids at position 3 of the internal beta sheet are alanine. Preferably, if alanine is not present the amino acid is glycine.

When the polypeptide is closely related to SEQ ID NO:11 or SEQ ID NO:12 it is preferred that at least 60% of the amino acids at position 4 of the internal beta sheet are serine. Preferably, if serine is not present the amino acid is threonine, alanine or valine.

When the polypeptide is closely related to SEQ ID NO:11 or SEQ ID NO:12 it is preferred that the polypeptide comprises a single region of a 16 amino acid contiguous repeat. Preferably, the region is at least 20 repeats, more preferably at least 25 repeats. In an alternate embodiment, the region is about 29 repeats. Preferably, the repeats of the region comprise the sequence:

| | |
|---|---|
| X₁SX₁₃AX₁₄AX₂KX₁₅X₁₆X₃X₇AX₁₇SX₁₈. | (SEQ ID NO:34) |

### Where,

### X₁=Gly, Ser or Asn,

### X₂=Ser or Thr

### X₃=Ala or Ser

### X₇=Ser or Gly

### X₁₃=Ser, Val or Thr

### X₁₄=Ser, Gly, Thr, Val, or Ala

### X₁₅=Gly or Asp

### X₁₆=Ser, Ala or Gly

### X₁₇=Ser, Thr or Ala, and

### X₁₈=Gly, Asn, Gln or Asp.

In a preferred embodiment, at least 50%, more preferably at least 75% of residues in the internal beta sheet positions are small residues (alanine, glycine or serine).

In a further preferred embodiment, the polypeptide comprises a cysteine within about 20 amino acids of the N-terminus and/or C-terminus of the mature protein (namely, without a signal sequence).

For polypeptides closely related to any one of SEQ ID NO's 1 to 9, preferably the polypeptide comprises a cysteine within about 5 amino acids of the N-terminus and/or C-terminus of the mature protein. More preferably, the polypeptide comprises a cysteine at the N-terminus and/or C-terminus of the mature protein. Even more preferably, the polypeptide comprises a cysteine at the N-terminus and C-terminus of the mature protein.

Further guidance regarding amino acid substitutions which can be made to the polypeptides of the invention is provided in Tables 3 to 6. Where a predicted useful amino acid substitution based on the experimental data provided herein is in anyway in conflict with the exemplary substitutions provided in Table 1 it is preferred that a substitution based on the experimental data is used.

Polypeptides (and polynucleotides) of the invention can be purified (isolated) from a wide variety of Neuropteran and Coleopteran species and some Dipteran and Hymenopteran species. Examples of Neuropterans include, but are not limited to, any species of the Families Chrysopidae (green lacewing), Sisyridae (spongillaflies), Berothidae (beaded lacewings), Mantispidae (mantidflies) and Nymphidae (split footed lacewings). Examples of Coleoptera include, but are not limited to, species from the superfamily Cucujiformia (weevils) and the family Hydrophilidae (water beetles). Examples from Dipteran species include the genus Arachnocampa (glow worms) and examples from Hymenoptera include the tribe Nematini (sawflies). Such further polypeptides (and polynucleotides) can be characterized using the same procedures described herein for silks from Mallada signata.

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Oligonucleotides and/or polynucleotides of the invention hybridize to a silk gene of the present invention, or a region flanking said gene, under stringent conditions. The term “stringent hybridization conditions” and the like as used herein refers to parameters with which the art is familiar, including the variation of the hybridization temperature with length of an oligonucleotide. Nucleic acid hybridization parameters may be found in references which compile such methods, Sambrook, et al. (supra), and Ausubel, et al. (supra). For example, stringent hybridization conditions, as used herein, can refer to hybridization at 65° C. in hybridization buffer (3.5×SSC, 0.02% Ficoll, 0.02% polyvinyl pyrrolidone, 0.02% Bovine Serum Albumin (BSA), 2.5 mM NaH₂PO₄ (pH7), 0.5% SDS, 2 mM EDTA), followed by one or more washes in 0.2.×SSC, 0.01% BSA at 50° C. Alternatively, the nucleic acid and/or oligonucleotides (which may also be referred to as “primers” or “probes”) hybridize to the region of the an insect genome of interest, such as the genome of a honeybee, under conditions used in nucleic acid amplification techniques such as PCR.

Oligonucleotides of the present invention can be RNA, DNA, or derivatives of either. Although the terms polynucleotide and oligonucleotide have overlapping meaning, oligonucleotides are typically relatively short single stranded molecules. The minimum size of such oligonucleotides is the size required for the formation of a stable hybrid between an oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length.

Usually, monomers of a polynucleotide or oligonucleotide are linked by phosphodiester bonds or analogs thereof to form oligonucleotides ranging in size from a relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

The present invention includes oligonucleotides that can be used as, for example, probes to identify nucleic acid molecules, or primers to produce nucleic acid molecules. Oligonucleotides of the present invention used as a probe are typically conjugated with a detectable label such as a radioisotope, an enzyme, biotin, a fluorescent molecule or a chemiluminescent molecule.

### Recombinant Vectors

One embodiment of the present invention includes a recombinant vector, which comprises at least one isolated polynucleotide molecule of the present invention, inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises a polynucleotide molecule of the present invention operatively linked to an expression vector. The phrase operatively linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, animal, and plant cells. Particularly preferred expression vectors of the present invention can direct gene expression in plants cells. Vectors of the invention can also be used to produce the polypeptide in a cell-free expression system, such systems are well known in the art.

In particular, expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, plant or mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

Particularly preferred transcription control sequences are promoters active in directing transcription in plants, either constitutively or stage and/or tissue specific, depending on the use of the plant or parts thereof. These plant promoters include, but are not limited to, promoters showing constitutive expression, such as the 35S promoter of Cauliflower Mosaic Virus (CaMV), those for leaf-specific expression, such as the promoter of the ribulose bisphosphate carboxylase small subunit gene, those for root-specific expression, such as the promoter from the glutamine synthase gene, those for seed-specific expression, such as the cruciferin A promoter from Brassica napus, those for tuber-specific expression, such as the class-I patatin promoter from potato or those for fruit-specific expression, such as the polygalacturonase (PG) promoter from tomato.

Recombinant molecules of the present invention may also (a) contain secretory signals (i.e., signal segment nucleic acid sequences) to enable an expressed polypeptide of the present invention to be secreted from the cell that produces the polypeptide and/or (b) contain fusion sequences which lead to the expression of nucleic acid molecules of the present invention as fusion proteins. Examples of suitable signal segments include any signal segment capable of directing the secretion of a polypeptide of the present invention. Preferred signal segments include, but are not limited to, tissue plasminogen activator (t-PA), interferon, interleukin, growth hormone, viral envelope glycoprotein signal segments, Nicotiana nectarin signal peptide (U.S. Pat. No. 5,939,288), tobacco extensin signal, the soy oleosin oil body binding protein signal, Arabidopsis thaliana vacuolar basic chitinase signal peptide, as well as native signal sequences of a polypeptide of the invention. In addition, a nucleic acid molecule of the present invention can be joined to a fusion segment that directs the encoded polypeptide to the proteosome, such as a ubiquitin fusion segment. Recombinant molecules may also include intervening and/or untranslated sequences surrounding and/or within the nucleic acid sequences of the present invention.

### Host Cells

Another embodiment of the present invention includes a recombinant cell comprising a host cell transformed with one or more recombinant molecules of the present invention, or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides of the present invention or can be capable of producing such polypeptides after being transformed with at least one polynucleotide molecule of the present invention. Host cells of the present invention can be any cell capable of producing at least one protein of the present invention, and include bacterial, fungal (including yeast), parasite, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells. Particularly preferred host cells are plant cells such as those available from Deutsche Sammlung von Mikroorganismen and Zellkulturen GmbH (German Collection of Microorganisms and Cell Cultures).

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

### Transgenic Plants

The term “plant” refers to whole plants, plant organs (e.g. leaves, stems roots, etc), seeds, plant cells and the like. Plants contemplated for use in the practice of the present invention include both monocotyledons and dicotyledons. Target plants include, but are not limited to, the following: cereals (wheat, barley, rye, oats, rice, sorghum and related crops); beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape, mustard, poppy, olives, sunflowers, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers).

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

Constitutive plant promoters are well known. Further to previously mentioned promoters, some other suitable promoters include but are not limited to the nopaline synthase promoter, the octopine synthase promoter, CaMV 35S promoter, the ribulose-1,5-bisphosphate carboxylase promoter, Adh1-based pEmu, Act1, the SAM synthase promoter and Ubi promoters and the promoter of the chlorophyll a/b binding protein. Alternatively it may be desired to have the transgene(s) expressed in a regulated fashion. Regulated expression of the polypeptides is possible by placing the coding sequence of the silk protein under the control of promoters that are tissue-specific, developmental-specific, or inducible. Several tissue-specific regulated genes and/or promoters have been reported in plants. These include genes encoding the seed storage proteins (such as napin, cruciferin, 3-conglycinin, glycinin and phaseolin), zein or oil body proteins (such as oleosin), or genes involved in fatty acid biosynthesis (including acyl carrier protein, stearoyl-ACP desaturase, and fatty acid desaturases (fad 2-1)), and other genes expressed during embryo development (such as Bce4). Particularly useful for seed-specific expression is the pea vicilin promoter. Other useful promoters for expression in mature leaves are those that are switched on at the onset of senescence, such as the SAG promoter from Arabidopsis). A class of fruit-specific promoters expressed at or during anthesis through fruit development, at least until the beginning of ripening, is discussed in U.S. Pat. No. 4,943,674. Other examples of tissue-specific promoters include those that direct expression in tubers (for example, patatin gene promoter), and in fiber cells (an example of a developmentally-regulated fiber cell protein is E6 fiber).

Other regulatory sequences such as terminator sequences and polyadenylation signals include any such sequence functioning as such in plants, the choice of which would be obvious to the skilled addressee. The termination region used in the expression cassette will be chosen primarily for convenience, since the termination regions appear to be relatively interchangeable. The termination region which is used may be native with the transcriptional initiation region, may be native with the polynucleotide sequence of interest, or may be derived from another source. The termination region may be naturally occurring, or wholly or partially synthetic. Convenient termination regions are available from the Ti-plasmid of A. tumefaciens, such as the octopine synthase and nopaline synthase termination regions or from the genes for β-phaseolin, the chemically inducible lant gene, pIN.

Several techniques are available for the introduction of an expression construct containing a nucleic acid sequence encoding a polypeptide of interest into the target plants. Such techniques include but are not limited to transformation of protoplasts using the calcium/polyethylene glycol method, electroporation and microinjection or (coated) particle bombardment. In addition to these so-called direct DNA transformation methods, transformation systems involving vectors are widely available, such as viral and bacterial vectors (e.g. from the genus Agrobacterium). After selection and/or screening, the protoplasts, cells or plant parts that have been transformed can be regenerated into whole plants, using methods known in the art. The choice of the transformation and/or regeneration techniques is not critical for this invention.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Transgenic Non-Human Animals

Techniques for producing transgenic animals are well known in the art. A useful general textbook on this subject is Houdebine, Transgenic animals—Generation and Use (Harwood Academic, 1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Recovery Methods and Production of Silk

The silk proteins of the present invention may be extracted and purified from recombinant cells, such as plant, bacteria or yeast cells, producing said protein by a variety of methods. In one embodiment, the method involves removal of native cell proteins from homogenized cells/tissues/plants etc. by lowering pH and heating, followed by ammonium sulfate fractionation. Briefly, total soluble proteins are extracted by homogenizing cells/tissues/plants. Native proteins are removed by precipitation at pH 4.7 and then at 60° C. The resulting supernatant is then fractionated with ammonium sulfate at 40% saturation. The resulting protein will be of the order of 95% pure. Additional purification may be achieved with conventional gel or affinity chromatography.

In another example, cell lysates are treated with high concentrations of acid e.g. HCl or propionic acid to reduce pH to ˜1-2 for 1 hour or more which will solubilise the silk proteins but precipitate other proteins.

Fibrillar aggregates will form from solutions by spontaneous self-assembly of silk proteins of the invention when the protein concentration exceeds a critical value. The aggregates may be gathered and mechanically spun into macroscopic fibers according to the method of O'Brien et al. (I. O'Brien et al., “Design, Synthesis and Fabrication of Novel Self-Assembling Fibrillar Proteins”, in Silk Polymers: Materials Science and Biotechnology, pp. 104-117, Kaplan, Adams, Farmer and Viney, eds., c. 1994 by American Chemical Society, Washington, D.C.).

By nature of the inherent secondary structure, proteins of the invention will spontaneously form a cross-beta structure upon dehydration. As described below, the strength of the cross-beta structure can be enhanced through enzymatic or chemical cross-linking of lysine residues in close proximity.

Silk fibres and/or copolymers of the invention have a low processing requirement. The silk proteins of the invention require minimal processing e.g. spinning to form a strong fibre as they spontaneously forms strong cross-beta structures which can be reinforced with crosslinks such as lysine crosslinks. This contrasts with B. mori and spider recombinant silk polypeptides which require sophisticated spinning techniques in order to obtain the secondary structure (β-sheet) and strength of the fibre.

However, fibers may be spun from solutions having properties characteristic of a liquid crystal phase. The fiber concentration at which phase transition can occur is dependent on the composition of a protein or combination of proteins present in the solution. Phase transition, however, can be detected by monitoring the clarity and birefringence of the solution. Onset of a liquid crystal phase can be detected when the solution acquires a translucent appearance and registers birefringence when viewed through crossed polarizing filters.

In one fiber-forming technique, fibers can first be extruded from the protein solution through an orifice into methanol, until a length sufficient to be picked up by a mechanical means is produced. Then a fiber can be pulled by such mechanical means through a methanol solution, collected, and dried. Methods for drawing fibers are considered well-known in the art.

Further examples of methods which may be used for producing silk fibres and/or copolymers of the present are described in US 2004/0170827 and US 2005/0054830.

In a preferred embodiment, silk fibres and/or copolymers of the invention are crosslinked. In one embodiment, the silk fibres and/or copolymers are crosslinked to a surface/article/product etc of interest using techniques known in the art. In another embodiment (or in combination with the previous embodiment), at least some silk proteins in the silk fibres and/or copolymers are crosslinked to each other. Such crosslinking can be performed using chemical and/or enzymatic techniques known in the art. For example, enzymatic cross links can be catalysed by lysyl oxidase, whereas nonenzymatic cross links can be generated from glycated lysine residues (Reiser et al., 1992).

### Antibodies

The term “antibody” as used in this invention includes polyclonal antibodies, monoclonal antibodies, bispecific antibodies, diabodies, triabodies, heteroconjugate antibodies, chimeric antibodies including intact molecules as well as fragments thereof, such as Fab, F(ab′)2, and Fv which are capable of binding the epitopic determinant, and other antibody-like molecules.

Antibody fragments retain some ability to selectively bind with its antigen or receptor and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York (1988)).

(6) Single domain antibody, typically a variable heavy domain devoid of a light chain.

The phrase “specifically binds” means that under particular conditions, the compound binds a polypeptide of the invention and does not bind to a significant amount to other, for example, proteins or carbohydrates. Specific binding may require an antibody that is selected for its specificity. In another embodiment, an antibody is considered to “specifically binds” if there is a greater than 10 fold difference, and preferably a 25, 50 or 100 fold greater difference between the binding of the antibody to a polypeptide of the invention when compared to another protein, especially a silk protein.

As used herein, the term “epitope” refers to a region of a polypeptide of the invention which is bound by the antibody. An epitope can be administered to an animal to generate antibodies against the epitope, however, antibodies of the present invention preferably specifically bind the epitope region in the context of the entire polypeptide.

If polyclonal antibodies are desired, a selected mammal (e.g., mouse, rabbit, goat, horse, etc.) is immunised with an immunogenic polypeptide of the invention. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Other techniques for producing antibodies of the invention are known in the art.

Antibodies of the invention may be bound to a solid support and/or packaged into kits in a suitable container along with suitable reagents, controls, instructions and the like.

In an embodiment, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further, exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, for example, biotin. Such labeled antibodies can be used in techniques known in the art to detect polypeptides of the invention.

### Compositions

Compositions of the present invention may include an “acceptable carrier”. Examples of such acceptable carriers include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used.

In one embodiment, the “acceptable carrier” is a “pharmaceutically acceptable carrier”. The term pharmaceutically acceptable carrier refers to molecular entities and compositions that do not produce an allergic, toxic or otherwise adverse reaction when administered to an animal, particularly a mammal, and more particularly a human. Useful examples of pharmaceutically acceptable carriers or diluents include, but are not limited to, solvents, dispersion media, coatings, stabilizers, protective colloids, adhesives, thickeners, thixotropic agents, penetration agents, sequestering agents and isotonic and absorption delaying agents that do not affect the activity of the polypeptides of the invention. The proper fluidity can be maintained, for example, by the use of a coating, such as lecithin, by the maintenance of the required particle size in the case of dispersion and by the use of surfactants. More generally, the polypeptides of the invention can be combined with any non-toxic solid or liquid additive corresponding to the usual formulating techniques.

As outlined herein, in some embodiments a polypeptide, a silk fiber and/or a copolymer of the invention is used as a pharmaceutically acceptable carrier.

Other suitable compositions are described below with specific reference to specific uses of the polypeptides of the invention.

### Uses

Silk proteins are useful for the creation of new biomaterials because of their exceptional toughness and strength. However, the fibrous proteins of spiders and insects are generally large proteins (over 100 kDa) and consist of highly repetitive amino acid sequences. These proteins are encoded by large genes containing highly biased codons making them particularly difficult to produce in recombinant systems. By comparison, the silk proteins of the invention are short and less-repetitive. These properties make the genes encoding these proteins particularly attractive for recombinant production of new biomaterials.

The silk proteins, silk fibers and/or copolymers of the invention can be used for a broad and diverse array of medical, military, industrial and commercial applications. The fibers can be used in the manufacture of medical devices such as sutures, skin grafts, cellular growth matrices, replacement ligaments, and surgical mesh, and in a wide range of industrial and commercial products, such as, for example, cable, rope, netting, fishing line, clothing fabric, bullet-proof vest lining, container fabric, backpacks, knapsacks, bag or purse straps, adhesive binding material, non-adhesive binding material, strapping material, tent fabric, tarpaulins, pool covers, vehicle covers, fencing material, sealant, construction material, weatherproofing material, flexible partition material, sports equipment; and, in fact, in nearly any use of fiber or fabric for which high tensile strength and elasticity are desired characteristics. The silk proteins, silk fibers and/or copolymers of the present invention also have applications in compositions for personal care products such as cosmetics, skin care, hair care and hair colouring; and in coating of particles, such as pigments.

The silk proteins may be used in their native form or they may be modified to form derivatives, which provide a more beneficial effect. For example, the silk protein may be modified by conjugation to a polymer to reduce allergenicity as described in U.S. Pat. No. 5,981,718 and U.S. Pat. No. 5,856,451. Suitable modifying polymers include, but are not limited to, polyalkylene oxides, polyvinyl alcohol, poly-carboxylates, poly(vinylpyrolidone), and dextrans. In another example, the silk proteins may be modified by selective digestion and splicing of other protein modifiers. For example, the silk proteins may be cleaved into smaller peptide units by treatment with acid at an elevated temperature of about 60° C. The useful acids include, but are not limited to, dilute hydrochloric, sulfuric or phosphoric acids. Alternatively, digestion of the silk proteins may be done by treatment with a base, such as sodium hydroxide, or enzymatic digestion using a suitable protease may be used.

The proteins may be further modified to provide performance characteristics that are beneficial in specific applications for personal care products. The modification of proteins for use in personal care products is well known in the art. For example, commonly used methods are described in U.S. Pat. No. 6,303,752, U.S. Pat. No. 6,284,246, and U.S. Pat. No. 6,358,501. Examples of modifications include, but are not limited to, ethoxylation to promote water-oil emulsion enhancement, siloxylation to provide lipophilic compatibility, and esterification to aid in compatibility with soap and detergent compositions. Additionally, the silk proteins may be derivatized with functional groups including, but not limited to, amines, oxiranes, cyanates, carboxylic acid esters, silicone copolyols, siloxane esters, quaternized amine aliphatics, urethanes, polyacrylamides, dicarboxylic acid esters, and halogenated esters. The silk proteins may also be derivatized by reaction with diimines and by the formation of metal salts.

Consistent with the above definitions of “polypeptide” (and “protein”), such derivatized and/or modified molecules are also referred to herein broadly as “polypeptides” and “proteins”.

Silk proteins of the invention can be spun together and/or bundled or braided with other fiber types. Examples include, but are not limited to, polymeric fibers (e.g., polypropylene, nylon, polyester), fibers and silks of other plant and animal sources (e.g., cotton, wool, Bombyx mori, spider silk or honey bee (for example see, WO 2007/038837), and glass fibers. A preferred embodiment is silk fiber braided with 10% polypropylene fiber. The present invention contemplates that the production of such combinations of fibers can be readily practiced to enhance any desired characteristics, e.g., appearance, softness, weight, durability, water-repellant properties, improved cost-of-manufacture, that may be generally sought in the manufacture and production of fibers for medical, industrial, or commercial applications.

### Personal Care Products

Cosmetic and skin care compositions may be anhydrous compositions comprising an effective amount of silk protein in a cosmetically acceptable medium. The uses of these compositions include, but are not limited to, skin care, skin cleansing, make-up, and anti-wrinkle products. An effective amount of a silk protein for cosmetic and skin care compositions is herein defined as a proportion of from about 10⁻⁴ to about 30% by weight, but preferably from about 10⁻³ to 15% by weight, relative to the total weight of the composition. This proportion may vary as a function of the type of cosmetic or skin care composition. Suitable compositions for a cosmetically acceptable medium are described in U.S. Pat. No. 6,280,747. For example, the cosmetically acceptable medium may contain a fatty substance in a proportion generally of from about 10 to about 90% by weight relative to the total weight of the composition, where the fatty phase containing at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments.

Emulsified cosmetics and quasi drugs which are producible with the use of emulsified materials comprising at least one silk protein of the present invention include, for example, cleansing cosmetics (beauty soap, facial wash, shampoo, rinse, and the like), hair care products (hair dye, hair cosmetics, and the like), basic cosmetics (general cream, emulsion, shaving cream, conditioner, cologne, shaving lotion, cosmetic oil, facial mask, and the like), make-up cosmetics (foundation, eyebrow pencil, eye cream, eye shadow, mascara, and the like), aromatic cosmetics (perfume and the like), tanning and sunscreen cosmetics (tanning and sunscreen cream, tanning and sunscreen lotion, tanning and sunscreen oil, and the like), nail cosmetics (nail cream and the like), eyeliner cosmetics (eyeliner and the like), lip cosmetics (lipstick, lip cream, and the like), oral care products (tooth paste and the like) bath cosmetics (bath products and the like), and the like.

The cosmetic composition may also be in the form of products for nail care, such as a nail varnish. Nail varnishes are herein defined as compositions for the treatment and colouring of nails, comprising an effective amount of silk protein in a cosmetically acceptable medium. An effective amount of a silk protein for use in a nail varnish composition is herein defined as a proportion of from about 10⁻⁴ to about 30% by weight relative to the total weight of the varnish. Components of a cosmetically acceptable medium for nail varnishes are described in U.S. Pat. No. 6,280,747. The nail varnish typically contains a solvent and a film forming substance, such as cellulose derivatives, polyvinyl derivatives, acrylic polymers or copolymers, vinyl copolymers and polyester polymers. The composition may also contain an organic or inorganic pigment.

Hair care compositions are herein defined as compositions for the treatment of hair, including but not limited to shampoos, conditioners, lotions, aerosols, gels, and mousses, comprising an effective amount of silk protein in a cosmetically acceptable medium. An effective amount of a silk protein for use in a hair care composition is herein defined as a proportion of from about 10⁻² to about 90% by weight relative to the total weight of the composition. Components of a cosmetically acceptable medium for hair care compositions are described in US 2004/0170590, U.S. Pat. No. 6,280,747, U.S. Pat. No. 6,139,851, and U.S. Pat. No. 6,013,250. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants, as given above.

Hair colouring compositions are herein defined as compositions for the colouring, dyeing, or bleaching of hair, comprising an effective amount of silk protein in a cosmetically acceptable medium. An effective amount of a silk protein for use in a hair colouring composition is herein defined as a proportion of from about 10⁻⁴ to about 60% by weight relative to the total weight of the composition. Components of a cosmetically acceptable medium for hair colouring compositions are described in US 2004/0170590, U.S. Pat. No. 6,398,821 and U.S. Pat. No. 6,129,770. For example, hair colouring compositions generally contain a mixture of inorganic peroxygen-based dye oxidizing agent and an oxidizable coloring agent. The peroxygen-based dye oxidizing agent is most commonly hydrogen peroxide. The oxidative hair coloring agents are formed by oxidative coupling of primary intermediates (for example p-phenylenediamines, p-aminophenols, p-diaminopyridines, hydroxyindoles, aminoindoles, aminothymidines, or cyanophenols) with secondary intermediates (for example phenols, resorcinols, m-aminophenols, m-phenylenediamines, naphthols, pyrazolones, hydroxyindoles, catechols or pyrazoles). Additionally, hair colouring compositions may contain oxidizing acids, sequestrants, stabilizers, thickeners, buffers carriers, surfactants, solvents, antioxidants, polymers, non-oxidative dyes and conditioners.

The silk proteins can also be used to coat pigments and cosmetic particles in order to improve dispersibility of the particles for use in cosmetics and coating compositions. Cosmetic particles are herein defined as particulate materials such as pigments or inert particles that are used in cosmetic compositions. Suitable pigments and cosmetic particles, include, but are not limited to, inorganic color pigments, organic pigments, and inert particles. The inorganic color pigments include, but are not limited to, titanium dioxide, zinc oxide, and oxides of iron, magnesium, cobalt, and aluminium. Organic pigments include, but are not limited to, D&C Red No. 36, D&C Orange No. 17, the calcium lakes of D&C Red Nos. 7, 11, 31 and 34, the barium lake of D&C Red No. 12, the strontium lake D&C Red No. 13, the aluminium lake of FD&C Yellow No. 5 and carbon black particles. Inert particles include, but are not limited to, calcium carbonate, aluminium silicate, calcium silicate, magnesium silicate, mica, talc, barium sulfate, calcium sulfate, powdered Nylon™, perfluorinated alkanes, and other inert plastics.

The silk proteins may also be used in dental floss (see, for example, US 2005/0161058). The floss may be monofilament yarn or multifilament yarn, and the fibers may or may not be twisted. The dental floss may be packaged as individual pieces or in a roll with a cutter for cutting pieces to any desired length. The dental floss may be provided in a variety of shapes other than filaments, such as but not limited to, strips and sheets and the like. The floss may be coated with different materials, such as but not limited to, wax, polytetrafluoroethylene monofilament yarn for floss.

The silk proteins may also be used in soap (see, for example, US 2005/0130857).

### Pigment and Cosmetic Particle Coating

The effective amount of a silk protein for use in pigment and cosmetic particle coating is herein defined as a proportion of from about 10⁻⁴ to about 50%, but preferably from about 0.25 to about 15% by weight relative to the dry weight of particle. The optimum amount of the silk protein to be used depends on the type of pigment or cosmetic particle being coated. For example, the amount of silk protein used with inorganic color pigments is preferably between about 0.01% and 20% by weight. In the case of organic pigments, the preferred amount of silk protein is between about 1% to about 15% by weight, while for inert particles, the preferred amount is between about 0.25% to about 3% by weight. Methods for the preparation of coated pigments and particles are described in U.S. Pat. No. 5,643,672. These methods include: adding an aqueous solution of the silk protein to the particles while tumbling or mixing, forming a slurry of the silk protein and the particles and drying, spray drying a solution of the silk protein onto the particles or lyophilizing a slurry of the silk protein and the particles. These coated pigments and cosmetic particles may be used in cosmetic formulations, paints, inks and the like.

### Biomedical

The silk proteins may be used as a coating on a bandage to promote wound healing. For this application, the bandage material is coated with an effective amount of the silk protein. For the purpose of a wound-healing bandage, an effective amount of silk protein is herein defined as a proportion of from about 10⁻⁴ to about 30% by weight relative to the weight of the bandage material. The material to be coated may be any soft, biologically inert, porous cloth or fiber. Examples include, but are not limited to, cotton, silk, rayon, acetate, acrylic, polyethylene, polyester, and combinations thereof. The coating of the cloth or fiber may be accomplished by a number of methods known in the art. For example, the material to be coated may be dipped into an aqueous solution containing the silk protein. Alternatively, the solution containing the silk protein may be sprayed onto the surface of the material to be coated using a spray gun. Additionally, the solution containing the silk protein may be coated onto the surface using a roller coat printing process. The wound bandage may include other additives including, but not limited to, disinfectants such as iodine, potassium iodide, povidon iodine, acrinol, hydrogen peroxide, benzalkonium chloride, and chlorohexidine; cure accelerating agents such as allantoin, dibucaine hydrochloride, and chlorophenylamine malate; vasoconstrictor agents such as naphazoline hydrochloride; astringent agents such as zinc oxide; and crust generating agents such as boric acid.

The silk proteins of the present invention may also be used in the form of a film as a wound dressing material. The use of silk proteins, in the form of an amorphous film, as a wound dressing material is described in U.S. Pat. No. 6,175,053. The amorphous film comprises a dense and nonporous film of a crystallinity below 10% which contains an effective amount of silk protein. For a film for wound care, an effective amount of silk protein is herein defined as between about 1 to 99% by weight. The film may also contain other components including but not limited to other proteins such as sericin, and disinfectants, cure accelerating agents, vasoconstrictor agents, astringent agents, and crust generating agents, as described above. Other proteins such as sericin may comprise 1 to 99% by weight of the composition. The amount of the other ingredients listed is preferably below a total of about 30% by weight, more preferably between about 0.5 to 20% by weight of the composition. The wound dressing film may be prepared by dissolving the above mentioned materials in an aqueous solution, removing insolubles by filtration or centrifugation, and casting the solution on a smooth solid surface such as an acrylic plate, followed by drying.

The silk proteins of the present invention may also be used in sutures (see, for example, US 2005/0055051). Such sutures can feature a braided jacket made of ultrahigh molecular weight fibers and silk fibers. The polyethylene provides strength. Polyester fibers may be woven with the high molecular weight polyethylene to provide improved tie down properties. The silk may be provided in a contrasting color to provide a trace for improved suture recognition and identification. Silk also is more tissue compliant than other fibers, allowing the ends to be cut close to the knot without concern for deleterious interaction between the ends of the suture and surrounding tissue. Handling properties of the high strength suture also can be enhanced using various materials to coat the suture. The suture advantageously has the strength of Ethibond No. 5 suture, yet has the diameter, feel and tie-ability of No. 2 suture. As a result, the suture is ideal for most orthopedic procedures such as rotator cuff repair, Achilles tendon repair, patellar tendon repair, ACL/PCL reconstruction, hip and shoulder reconstruction procedures, and replacement for suture used in or with suture anchors. The suture can be uncoated, or coated with wax (beeswax, petroleum wax, polyethylene wax, or others), silicone (Dow Corning silicone fluid 202A or others), silicone rubbers, PBA (polybutylate acid), ethyl cellulose (Filodel) or other coatings, to improve lubricity of the braid, knot security, or abrasion resistance, for example.

The silk proteins of the present invention may also be used in stents (see, for example, US 2004/0199241). For example, a stent graft is provided that includes an endoluminal stent and a graft, wherein the stent graft includes silk. The silk induces a response in a host who receives the stent graft, where the response can lead to enhanced adhesion between the silk stent graft and the host's tissue that is adjacent to the silk of the silk stent graft. The silk may be attached to the graft by any of various means, e.g., by interweaving the silk into the graft or by adhering the silk to the graft (e.g., by means of an adhesive or by means of suture). The silk may be in the form of a thread, a braid, a sheet, powder, etc. As for the location of the silk on the stent graft, the silk may be attached only the exterior of the stent, and/or the silk may be attached to distal regions of the stent graft, in order to assist in securing those distal regions to neighbouring tissue in the host. A wide variety of stent grafts may be utilized within the context of the present invention, depending on the site and nature of treatment desired. Stent grafts may be, for example, bifurcated or tube grafts, cylindrical or tapered, self-expandable or balloon-expandable, unibody or, modular, etc.

In addition to silk, the stent graft may contain a coating on some or all of the silk, where the coating degrades upon insertion of the stent graft into a host, the coating thereby delaying contact between the silk and the host. Suitable coatings include, without limitation, gelatin, degradable polyesters (e.g., PLGA, PLA, MePEG-PLGA, PLGA-PEG-PLGA, and copolymers and blends thereof), cellulose and cellulose derivatives (e.g., hydroxypropyl cellulose), polysaccharides (e.g., hyaluronic acid, dextran, dextran sulfate, chitosan), lipids, fatty acids, sugar esters, nucleic acid esters, polyanhydrides, polyorthoesters and polyvinylalcohol (PVA). The silk-containing stent grafts may contain a biologically active agent (drug), where the agent is released from the stent graft and then induces an enhanced cellular response (e.g., cellular or extracellular matrix deposition) and/or fibrotic response in a host into which the stent graft has been inserted.

The silk proteins of the present invention may also be used in a matrix for producing ligaments and tendons ex vivo (see, for example, US 2005/0089552). A silk-fiber-based matrix can be seeded with pluripotent cells, such as bone marrow stromal cells (BMSCs). The bioengineered ligament or tendon is advantageously characterized by a cellular orientation and/or matrix crimp pattern in the direction of applied mechanical forces, and also by the production of ligament and tendon specific markers including collagen type I, collagen type III, and fibronectin proteins along the axis of mechanical load produced by the mechanical forces or stimulation, if such forces are applied. In a preferred embodiment, the ligament or tendon is characterized by the presence of fiber bundles which are arranged into a helical organization. Some examples of ligaments or tendons that can be produced include anterior cruciate ligament, posterior cruciate ligament, rotator cuff tendons, medial collateral ligament of the elbow and knee, flexor tendons of the hand, lateral ligaments of the ankle and tendons and ligaments of the jaw or temporomandibular joint. Other tissues that may be produced by methods of the present invention include cartilage (both articular and meniscal), bone, muscle, skin and blood vessels.

The silk proteins of the present invention may also be used in hydrogels (see, for example, US 2005/0266992). Silk fibroin hydrogels can be characterized by an open pore structure which allows their use as tissue engineering scaffolds, substrate for cell culture, wound and burn dressing, soft tissue substitutes, bone filler, and as well as support for pharmaceutical or biologically active compounds.

The silk proteins may also be used in dermatological compositions (see, for example, US 2005/0019297). Furthermore, the silk proteins of the invention and derivatives thereof may also be used in sustained release compositions (see, for example, US 2004/0005363).

### Textiles

The silk proteins of the present invention may also be applied to the surface of fibers for subsequent use in textiles. This provides a monolayer of the protein film on the fiber, resulting in a smooth finish. U.S. Pat. No. 6,416,558 and U.S. Pat. No. 5,232,611 describe the addition of a finishing coat to fibers. The methods described in these disclosures provide examples of the versatility of finishing the fiber to provide a good feel and a smooth surface. For this application, the fiber is coated with an effective amount of the silk protein. For the purpose of fiber coating for use in textiles, an effective amount of silk protein is herein defined as a proportion of from about 1 to about 99% by weight relative to the weight of the fiber material. The fiber materials include, but are not limited to textile fibers of cotton, polyesters such as rayon and Lycra™, nylon, wool, and other natural fibers including native silk. Compositions suitable for applying the silk protein onto the fiber may include co-solvents such as ethanol, isopropanol, hexafluoranols, isothiocyanouranates, and other polar solvents that can be mixed with water to form solutions or microemulsions. The silk protein-containing solution may be sprayed onto the fiber or the fiber may be dipped into the solution. While not necessary, flash drying of the coated material is preferred. An alternative protocol is to apply the silk protein composition onto woven fibers. An ideal embodiment of this application is the use of silk proteins to coat stretchable weaves such as used for stockings.

### Composite Materials

Silk fibres can be added to polyurethane, other resins or thermoplastic fillers to prepare panel boards and other construction material or as moulded furniture and benchtops that replace wood and particle board. The composites can be also be used in building and automotive construction especially rooftops and door panels. The silk fibres re-enforce the resin making the material much stronger and allowing lighterweight construction which is of equal or superior strength to other particle boards and composite materials. Silk fibres may be isolated and added to a synthetic composite-forming resin or be used in combination with plant-derived proteins, starch and oils to produce a biologically-based composite materials. Processes for the production of such materials are described in JP 2004284246, US 2005175825, U.S. Pat. No. 4,515,737, JP 47020312 and WO 2005/017004.

### Paper Additives

The fibre properties of the silk of the invention can add strength and quality texture to paper making. Silk papers are made by mottling silk threads in cotton pulp to prepare extra smooth handmade papers is used for gift wrapping, notebook covers, carry bags. Processes for production of paper products which can include silk proteins of the invention are generally described in JP 2000139755.

### Advanced Materials

Silks of the invention have considerable toughness and stands out among other silks in maintaining these properties when wet (Hepburn et al., 1979).

Areas of substantial growth in the clothing textile industry are the technical and intelligent textiles. There is a rising demand for healthy, high value functional, environmentally friendly and personalized textile products. Fibers, such as those of the invention, that do not change properties when wet and in particular maintain their strength and extensibility are useful for functional clothing for sports and leisure wear as well as work wear and protective clothing.

Developments in the weapons and surveillance technologies are prompting innovations in individual protection equipments and battle-field related systems and structures. Besides conventional requirements such as material durability to prolonged exposure, heavy wear and protection from external environment, silk textiles of the invention can be processed to resist ballistic projectiles, fire and chemicals. Processes for the production of such materials are described in WO 2005/045122 and US 2005268443.

### EXAMPLES

### Example 1

### Identification of Abundant and Less Abundant Cross Beta Silk Genes MalXBFibroin and MalXBsFib

The colleterial gland was dissected from adult females from the green lacewing species Mallada signata (Neuroptera: Chrysopidae: Chrysopinae: Chrysopini: Mallada), a widespread species that is endemic to Australia and is the only member of the genus in Australia. The gland was stored in RNAlater (Ambion) and stored at 4° C. Total RNA (2.5 μg) was isolated from the colleterial gland with the RNAqueous4PCR kit (Ambion, Austin, Tex.), from which messenger RNA (mRNA) was isolated with the Micro-FastTrack™ 2.0 mRNA Isolation kit (Invitrogen, Calsbad, Calif.).

The cDNA library was constructed from the mRNA using the CloneMiner™ cDNA kit (Invitrogen, Calsbad, Calif., USA) with modifications from the standard protocol as described in Sutherland et al. (2006). The cDNA library comprised approximately 1×10⁶ colony forming units (cfu). However the vast majority of these contained vector sequences. The poor quality of the library was due to the minute amounts of tissue in the colleterial gland.

Approximately 3000 clones were screened on agar plates containing either kanamycin (kan) or chloramphincol (cmp). Although it was expected that clones with inserts would be cmp susceptible and kan resistant, it was found that the majority of the 1060 clones identified by this screening (cmp−, kan+) contained vector inserts.

PCR screening was conducted on these 1060 clones using primers to the vector sequence that would have been replaced if an insert were present. This screen identified 428 clones that did not contain the vector sequence identified by the PCR method. DNA was isolated from each of these clones and the size of the inserts was determined by restriction enzyme digestion. Sequence data was obtained from 99 of the clones containing inserts over 100 bp. The majority (85) of these clones contained fragments of the original vector.

Eight sequences contained short open reading frames (less than 100 amino acids) with no homology to each other or to sequences on the NCBI databases. The remaining sequences contained 7 copies of the silk gene MalXBFibroin and 1 copy of the silk gene MalXBsFib.

Two cDNAs (3204 and 3378 by open reading frames) were assumed to contain the coding region of the entire silk gene MalXBFibroin as they contained identical 5′ sequences that encoded a methionine residue followed by a signal peptide predicted by the algorithm SignalP 3.0 (Bendtsen et al., 2004) and extensive 3′ polyA tails. The other five identified MalXBFibroin cDNA contained partial sequences ranging from 795-2907 by in length with open reading frames ranging from 777-2181 by in length. The different MalXBFibroin cDNAs contain 84 single nucleotide polymorphisms relative to each other but encode only seven non-synonymous changes. Some clones have insertions and/or deletions.

The seven MalXBFibroins have been termed 1-7 herein, and provided as SEQ ID NO's 1 to 9 (MalXBFibroin1 and 2 with and without the signal sequence). Corresponding encoding cDNAs are provided as SEQ ID NO's 12 to 20.

The copy of the MalXBsFib gene contained a signal peptide and a polyA tail, so was assumed to be complete. The MalXBsFib protein with and without the signal sequence is provided as SEQ ID NO's 10 and 11 respectively, whilst the encoding cDNAs are provided as SEQ ID NO's 21 and 22 respectively.

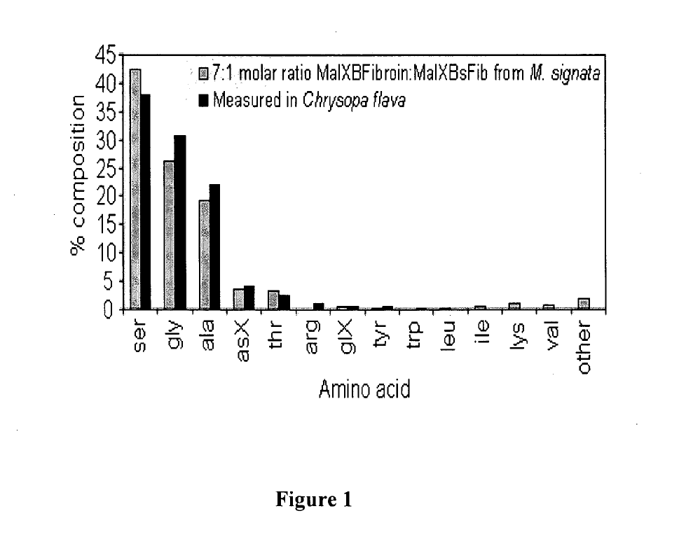

The amino acid composition of the egg stalk proteins encoded by MalXBFibroin and MalXBsFib were particularly high in serine (MalXBFibroin: 43%; MalXBsFib: 34%) and were high in glycine (MalXBFibroin: 27%; MalXBsFib: 21%) and alanine (MalXBFibroin: 19%; MalXBsFib: 26%). The inventors found 7 copies of MalXBFibroin cDNA and a single copy of MalXBsFib cDNA. A molar ratio of amino acids predicted from this (7:1) ratio of the egg stalk proteins was very similar to the composition measured previously in the egg stalk silk of Chrysopa flava, a related lacewing species (Lucas et al., 1957; FIG. 1).

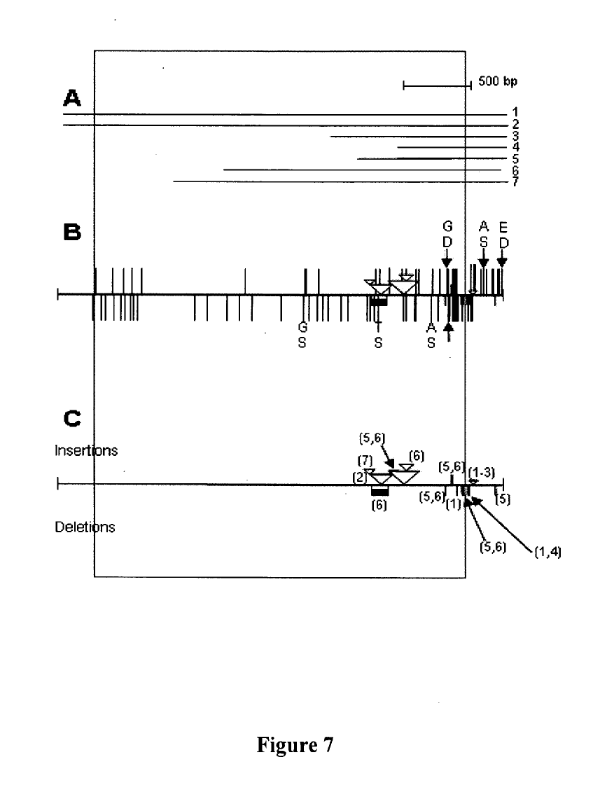

The silk gene sequence MalXBFibroin contained regions of repetitive sequence. Alignment with the minimal number of mutation events between the cDNA contained six insertions (6-289 bp), six deletions (6-336 bp) and 84 single nucleotide polymorphisms (FIG. 7B; total of 96 mutation events). All other alignments increased the number of mutation events. This alignment suggests that each of the 7 cDNA have a slightly different architecture (FIG. 7C).

Blastp of MalXBFibroin against the NCBI non-redundant database found no significant hits (best expect=0.58). Blastp of MalXBsFib found hits to a family of bacterial ice-nucleation proteins (best expect=2e-9). Blast 2 Sequence found no relationship between MalXBFibroin and MalXBsFib. It is barely conceivable that Mallada signata insects acquired the MalXBsFib gene by horizontal transfer from bacteria, but more likely that the relatedness found is due to both the MalXBsFib protein and the ice-nucleation protein containing an extensive region of repeat motifs with the same length (MalXBsFib has 16-mer repeats, as described below; the bacterial protein has octamers, which also make 16-mer repeats). This means that a few random amino acids identities between the different repeat units would become many identities along the full lengths of the proteins.

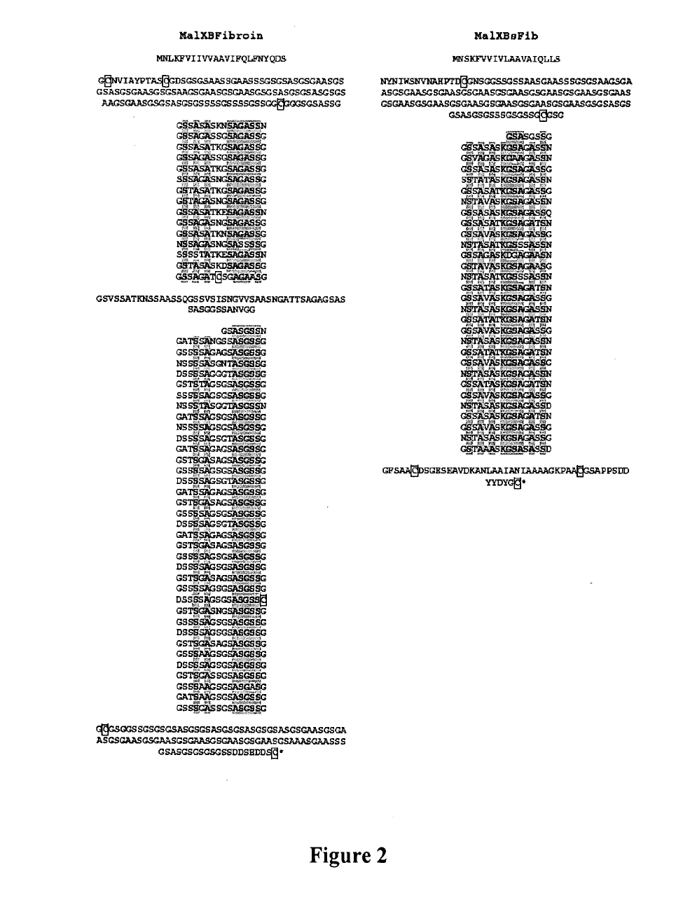

The primary architectures of MalXBFibroin and MalXBsFib are shown in FIG. 2. Both proteins begin with a signal peptide for secretion (predicted by SignalP 3.0), as expected for silks. The mature MalXBFibroin sequence is flanked by moderate-length N-terminal and C-terminal regions with a slightly repetitive character. The mature MalXBsFib is flanked by a moderate-length N-terminal region with a slightly repetitive character and a short non-repetitive C-terminal region. The bulk of both proteins is made up of repetitive regions with 16-residue motifs. Approximately half of the residues in the repeat motifs are conserved (grey shading in FIG. 2). The repeat motifs of MalXBFibroin are interrupted by one short non-repetitive sequence. The two divided repeat regions together make up 73% of the mature protein sequence of MalXBFibroin. The repeat region of MalXBsFib is continuous and comprises 70% of the mature protein sequence.

The seven different clones of MalXBFibroin that were found in the colleterial silk gland cDNA library have a variety of insertions and deletions. The sizes of the four deletions that occur within the region encoding the repeat motifs of MalXBFibroin are all multiples of 48 base pairs, which is equivalent to 16 residues. This supports the idea that 16-residue periodicity is important to the function of the Mallada signata egg stalk silk proteins.

Mallada signata egg stalk silk has a cross-beta protein structure, as described below. The 16-residue periodicity found in the MalXBFibroin and MalXBsFib proteins imply a 16-residue structural repeat in the cross-beta structure, which equates to 8-residue length beta strands consistent with wide angle X-ray scattering data obtained from single Mallada signata egg stalks (see below).

Both proteins contain a number of cysteine residues. MalXBFibroin contains a signal peptide immediately followed by several cysteine residues. Cysteine residues are also found in four positions approximately evenly spaced throughout the cross beta section, and an additional cysteine is found as the last residue in the protein. MalXBsFib contains a signal peptide immediately followed by a single cysteine residue. Cysteine residues are found at either end of the cross beta section and two additional cysteines are found in the C-termini with one being the last residue in the protein. It is likely that at least some of these cysteine residues are responsible for inter chain bonds that increase the relative size of the silk protein. This is important because of the relatively small size of the individual proteins (MalXBFibroin: 85-90 KDa; MalXBsFib: 57.4 KDa).

### Example 2

### The Silk has a Quarter-Staggered Cross-Beta Protein Structure

Wide angle X-ray scattering (WAXS) measurements were performed at the high-flux ChemMatCARS beamline, Sector 15 of the Advanced Photon Source (Cookson et al., 2006). Individual Mallada signata egg stalks were mounted in air, perpendicular to the beam, with WAXS patterns collected in transmission. An optical microscope alignment system was used to accurately position samples in the X-ray beam.

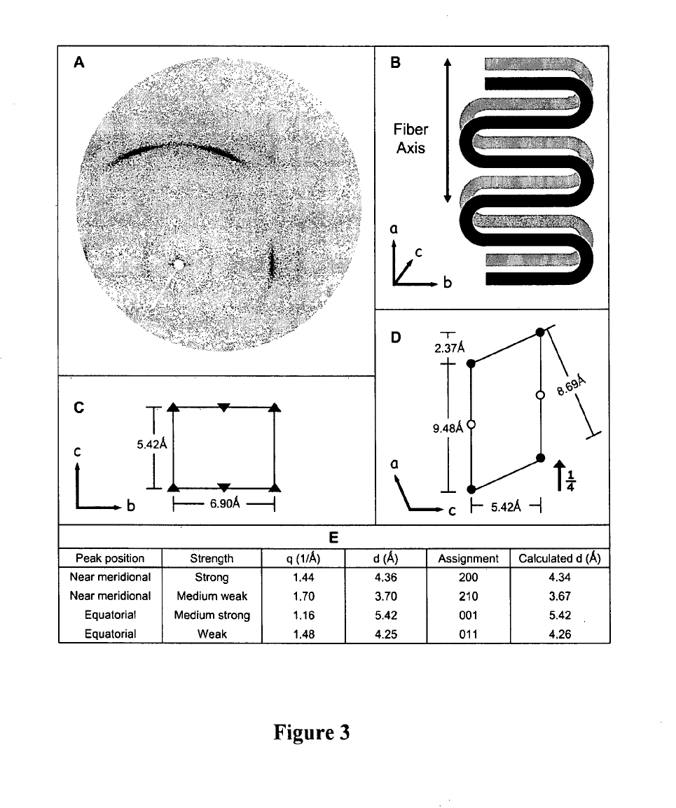

The protein structure of single Mallada signata egg stalks was probed by synchrotron wide-angle x-ray scattering (FIG. 3A). The main scattering peak positions corresponded to a beta-sheet structure. With the egg stalk vertical in the x-ray beam, the strong reflection attributable to inter-strand spacing (˜0.47 nm) was in a near-meridional position. This indicates a cross-beta structure with stacked beta-strands running perpendicular to the direction of the fibre (FIG. 3B), rather than an extended beta sheet with long strands running parallel to the fibre direction.

A strong peak on the equator of the scattering pattern is attributable to inter-sheet scattering. The inter-sheet distance of the egg stalk silk is 0.542 nm, which is similar to the beta-sheet spacing of polyserine (0.54-0.55 nm). This is consistent with the serine-rich composition of green lacewing egg stalk silk (FIG. 1).

No primary peak is observed that corresponds to the structural repeats along the length of the beta strands (˜0.69 nm). If a beta strand within the cross-beta structure is eight residues long, including a turn, it would contain only three 2-residue structural repeats. This number of repeats would not be expected to produce a reflection of detectable strength.

The reflection attributable to inter-strand spacing was found in a near-meridional rather than meridional position. This implies that the unit cell for Mallada signata egg stalk silk is not orthogonal. We constructed a pseudo cell for a quarter staggered cross-beta protein structure (FIGS. 3B, 3C, 3D). Calculations of scattering from this pseudo cell could predict all peaks found on the experimental scattering pattern with good agreement (FIG. 3E).

The main features of the wide-angle x-ray scattering pattern of Mallada signata egg stalk silk are much like the scattering from dry Chrysopa flava egg stalks (Parker and Rudall, 1957; Geddes et al., 1968). Our proposed quarter-staggered cross-beta protein structure is similar to the model of Geddes et al. (1968) except that we have not observed evidence for a departure from exact quarter-staggering as reported by these authors.

### Example 3

### A Highly Regular Sequence-Structure Model can be Constructed

The present inventors constructed a model for the Mallada signata egg stalk silk by fitting the repeat sequences with 16-residue periodicity of MalXBFibroin and MalXBsFibroin to an 8-residue strand cross-beta structure. Assuming regular four-residue beta turns between strands, for each repeat sequence there was a choice of eight positions where beta-turns could be started. The inventors selected the beta-turn positions which placed as many as possible of the bulky or charged amino acids as one of the middle two residues of a beta turn. Isolated bulky or charged residues located in a beta-strand would disrupt the interactions between stacked beta-sheets, therefore it is energetically more favourable for their side chains to be extended out from the sheets as part of a beta-turn. The sequence-structure model is shown in FIG. 4. All of the bulky or charged amino acids (bold letters) are placed in turns, and all but one are in the middle two residues of these turns.

The side chains of amino acids in a beta sheet project from the two faces of the sheet alternately, with the exception of amino acids in the middle two residues of a beta turn, whose side chains extend in the protein chain direction. The sequence-structure model predicts that the opposing faces of the cross-beta sheets formed by Mallada signata egg stalk silk have different amino acid side chain compositions (Table 2) and thus slightly differing properties. One side of the MalXBFibroin beta sheet is very rich in serine and also contains some threonine; this face is capable of extensive hydrogen bonding with an adjacent sheet. The other side is rich in both alanine and serine so is more hydrophobic. The average side chain volume of the two faces is similar (Table 2) and close to the side chain volume of a serine residue (18 cm³/mol). This is consistent with the x-ray scattering results which found uniform intersheet spacing of the same magnitude as in polyserine. The MalXBsFib beta sheet has one side that is very rich in serine, contains some threonine and a small amount of valine; this face has many potential hydrogen bonds and is comparatively bulky. The other side is very rich in alanine and moderately rich in serine so would be quite hydrophobic. The difference in side chain volume between the two faces (Table 2) might be sufficiently large that the x-ray scattering should predict non-uniform intersheet spacing. However, as MalXBsFib is much less abundant than MalXBFibroin in the silk, probably the signal from the more abundant protein would drown out the fine details of the signal from MalXBsFib.

TABLE 2
Amino acid side chain compositions of opposing beta sheet
faces of Mallada signata egg stalk silk proteins.

| | | | | | | Average side |
|---|---|---|---|---|---|---|
| | Ser | Ala | Gly | Thr | Val | chain volume^{#} |
| | (%) | (%) | (%) | (%) | (%) | (cm³/mol) |
| MalXBFibroin | 69 | 2 | 18 | 11 | — | 16.5 |
| face 1 | | | | | | |
| MalXBFibroin | 44 | 45 | 12 | — | — | 15 |
| face 2 | | | | | | |
| MalXBsFib face 1 | 60 | 2 | 16 | 16 | 5 | 19 |
| MalXBsFib face 2 | 34 | 65 | 1 | — | — | 16.5 |

^{#}Calculated relative to glycine from Harpaz et al. (1994).

The internal beta sheet residues found in the model of MalXBFibroin and MalXBsFib are most commonly serine, alanine and glycine with lower amounts of the slightly larger residues threonine and valine (Tables 3 and 4). Traces of cysteine, isoleucine and asparagine are also present. The beta sheets of the MalXBFibroin silk model are relatively symmetric with serine (the most abundant residue in the internal beta sheet positions, Table 3) side chains comprising 68% of the side chains on one side (side A) of the sheet and 44% on the other (side B). The side chain spacing of the sheets in the Chrysopa flava egg stalk silk was previously measured at 5.45 Å which is consistent with an abundance of serine (Geddes et al., 1968). Side A also contains 11.1% of threonine and valine, both residues that are larger than serine, while side B contains only 1.2% threonine and valine. MalXBsFib is less symmetrical with one side (side A) containing 60.6% serine and 20.6% threonine and valine, whereas the other side (side B) contains 34.4% serine and no larger residues.

TABLE 3
Amino acids in each of the internal beta sheet positions of MalXBFibroin.

| | Internal beta sheet position | | | |
|---|---|---|---|---|
| | 1 | 2 | 3 | 4 |
| Overall | Ala (92.9) | Ser (65.2) | Ser (35.7) | Ser (78.6) |
| | | | Gly (33.9) | |
| | Ser (1.8) | Gly (27.7) | Ala (28.6) | Thr (16.1) |
| | Gly (1.8) | Ala (3.6) | | Ala (3.6) |
| | Val (1.8) | Thr (2.7) | Val (0.9) | Gly (0.9) |
| | Cys (0.9) | Asn (0.9) | Thr (0.9) | Val (0.9) |
| | Ile (0.9) | | | |
| Top section | Ala (90.6) | Gly (68.8) | Ala (90.6) | Ser (87.5) |
| | | Ser (28.1) | | |
| | Ser (3.1) | | Ser (6.3) | Thr (9.4) |
| | Cys (3.1) | Thr (3.1) | Gly (3.1) | Ala (3.1) |
| | Gly (3.1) | | | |
| Middle section | Ala (55.6) | Ser (77.8) | Ser (33.3) | Ser (44.4) |
| | Val (22.2) | Ala (11.1) | Ala (33.3) | Thr (22.2) |
| | Ile (11.1) | Asn (11.1) | Val (11.1) | Ala (22.2) |
| | Ser (11.1) | | Thr (11.1) | Val (11.1) |
| | | | Gly (11.1) | |
| Bottom section | Ala (98.6) | Ser (80.3) | Gly (50.7) | Ser (78.9) |
| | | | Ser (49.3) | Thr (18.3) |
| | Gly (1.4) | Gly (12.7) | | |
| | | Ala (4.2) | | Ala (1.4) |
| | | Thr (2.8) | | Gly (1.4) |

Numbers in brackets indicate percentage of the residue in that position. Calculated in all the beta sheet positions (overall: 112 strands); in the first 32 strands; in the nine strands in the middle section of the structure (see FIG. 4), and in the last 71 strands.

TABLE 4
Amino acids in each of the internal beta sheet positions of MalXBsFib.
Internal beta sheet position

| 1 | 2 | 3 | 4 |
|---|---|---|---|
| Ala (95.0) | Gly (46.7) | Ala (96.7) | Ser (66.7) |
| | Ser (23.3) | | Thr (28.3) |
| | Val (13.3) | | |
| | Thr (8.3) | | |
| Ser (3.3) | Ala (1.7) | Gly (3.3) | Ala (3.2) |
| Gly (1.7) | Cys (1.7) | | Val (1.7) |

Numbers in brackets indicate percentage of the residue in that position. Calculated in all the beta sheet positions (60 strands).

Indels identified in MalXBFibroin encode insertions or deletions of complete eight-residue beta strands and in no case does an insertion or deletion disrupt a beta sheet strand.

The selection of beta-turn positions was tested by comparison with beta-turn prediction by the COUDES algorithm (Fuchs and Alix, 2005). The algorithm predicted 76% of the beta-turns selected for the first repetitive region of MalXBFibroin, 83% of the beta-turns selected for the second repetitive region of MalXBFibroin, and 76% of the beta-turns selected for the repetitive region of MalXBsFib. Any other choice of beta-turn positioning gave a significantly lower prediction rate. The types of beta-turn predicted are shown in FIG. 4 (Roman numerals). The MalXBFibroin repeat regions mainly had type II beta-turns on one side of the sheet, with the mirror image type II′ turns on the other side. MalXBsFib had primarily type II turns on one side of the sheet with type I′ turns on the other side. Only the common type II, II′ and I′ turns were predicted; none of the more exotic turns. Generally beta turns of the kind predicted in the silk proteins have a propensity for glycine in either the i+1 or i+2 turn positions (type I, I′ and II: i+2=glycine, type II′: i+1=glycine; EMBL EBI PDsum database). In the Chrysopa flava lacewing silk protein, glycine is predicted to be preferred over other residues in one of the i+1 or i+2 turn positions, as side groups other than hydrogen can only be accommodated in one turn position (Geddes et al., 1968). Another feature of the proteins is that virtually all the bulky and/or charged residues in the cross-beta sequences are located as the non-glycine residues of the turns (Tables 5 and 6).

Three distinct sections are present in the MalXBFibroin model (FIG. 4, Tables 3 and 5):
a) a top section that contained bulky residues (lysine, glutamic acid, isoleucine and glutamine) in turns and with an internal sheet pattern AGAS.
b) a nine sheet middle section that contained a higher proportion of moderately large residues, particularly valine, within the sheets.
c) a bottom section with an internal sheet pattern AS(G/S)S and no large residues.

The model is conservative in estimating the extent of cross-beta sheet structure and fits only the repetitive regions of MalXBFibroin and MalXBsFib. It is possible that a less regular cross-beta structure extends a short distance into the N or C termini regions of the sequences, or through all or part of the 56-residue (multiple of eight) interruption to the repetitive regions in MalXBFibroin. However even this conservative model predicts a high degree of crystallinity in the Mallada signata egg stalk silk proteins: 73% for MalXBFibroin and 70% for MalXBsFib. This is greater than the overall crystallinity of silkworm silk (˜40%; lizuka, 1970) due to the apparent absence of sericins in the lacewing egg stalk silk.

TABLE 5
Amino acids in each of the turn positions of MalXBFibroin.

| | Beta turn position number | | | |
|---|---|---|---|---|
| | i | i + 1 | i + 2 | i + 3 |
| Right side turn | Ser (100) | Gly (85.2) | Gly (72.7) | Ser (87.3) |
| | | Asn (11.1) | Asp (14.5) | Ala (12.7) |
| | | Cys (1.9) | Asn (7.3) | |
| | | Gln (1.9) | Ser (5.5) | |
| Left side turn | Gly (49.1) | Ser (41.8) | Gly (83.6) | Ser (85.5) |
| | Ser (34.5) | | | |
| | | Ala (18.2) | Asn (7.3) | Thr (10.9) |
| | Thr (14.5) | Lys (16.4) | Glu (3.6) | Gly (1.8) |
| | Asn (1.8) | Asn (10.9) | Ser (3.6) | Val (1.8) |
| | | Gly (10.9) | Asp (1.8) | |
| | | Cys (1.8) | | |

Right and left correspond to relative position of the turn in FIG. 4. Each turn type contains 54-55 turns.

TABLE 6
Amino acids in each of the turn positions of MalXBsFib.

| | Beta turn position number | | | |
|---|---|---|---|---|
| | i | i + 1 | i + 2 | i + 3 |
| Right side turn | Ser (100) | Asn (53.3) | Gly (70.0) | Ser (100) |
| | | Gly (36.7) | Asn (26.7) | |
| | | Asp (6.7) | Ser (3.3) | |
| | | Gln (3.3) | | |
| Left side turn | Ser (80.0) | Lys (96.7) | Gly (96.7) | Ser (93.3) |
| | Thr (20.0) | Gly (3.3) | Asp (3.3) | Ala (3.3) |
| | | | | Gly (3.3) |

Right and left correspond to relative position of the turn in FIG. 4. Each turn type contains 30 turns.

### Example 4

### Cross-Links in the Silk Contribute to its Mechanical Properties

The Mallada signata egg stalk silk proteins contain a number of cysteine residues (framed in FIG. 2). Five of the seven cysteines in MalXBFibroin are located in the N-terminal and C-terminal regions, while the other two are placed in the middle two residues of predicted beta-turns. All of the five cysteines in MalXBsFib are located in the N-terminal and C-terminal regions. As none of the cysteine residues are found in the interior of a beta sheet all of them are potentially able to form intramolecular or intermolecular cross-links.

Results of mechanical tests show different properties for egg stalks in reducing solutions (data not shown). This indicates the presence of extensive cystine cross-linking.

The lateral stiffness of Mallada signata egg stalks was measured by scanning probe microscopy in tapping mode, using silkworm silk as a control. The bending modulus of the egg stalks was calculated to be 90% greater than that of silkworm silk. The lateral stiffness of the Mallada signata silk is probably due to a structure involving cross-beta crystallites bound together by a three-dimensional network of cystine cross-links. A silk possessing high resistance to bending has obvious functional advantages for the insect. A rigid silk enables a very fine egg stalk, made at low metabolic cost, to suspend eggs safely out of the path of potential predators.

Experimental observations of the process of green lacewing egg laying indicate that the silk is initially secreted as a drop of liquid. The insect then pulls a thread from this drop which solidifies into an egg stalk within a few seconds (Duelli, 1984). We propose that the egg stalk silk is produced within the silk gland under reducing conditions, in which the cysteine residues are unmodified and the silk proteins are soluble. However when a fine thread of silk dope is exposed to air, oxidating conditions induce the formation of cystine cross-links and rapid hardening of the egg stalk into the mature silk.

The mechanical properties of a polymer are proportional to the molecular weight of the material (Donald and Windle, 1992). The extended beta silk proteins of spiders and silkworm are very large (>200 KDa) and consequently very strong—the mechanical properties of the lacewing silks are likely due to an enhanced relative size of the silks due to cysteine crosslinks.

### Example 5

### Expression of MalXBFibroin and MalXBsFib in Mallada signata

Primers for PCR analysis were designed using the Primer Express software of Applied Biosystems and chosen based on software scores and relative position on the gene. The sets used were (shown in 5′ to 3′ direction): MalXBFibroin forward primer—GTGCCGCTTCGAGCTCAG (SEQ ID NO:26); Reverse primer—ACTCCCTGTACACAGTTCAGC (SEQ ID NO:27) (110 nt fragment); MalXBsFib forward primer—ATAAAGCCAATCTTGCTGCCA (SEQ ID NO:28); Reverse primer—ACTCCCTGTACACAGTTCAGC (SEQ ID NO:29); Mallada signata elongation factor (Genbank accession number) forward primer—GGTACTGGTGAATTCGAAGC (SEQ ID NO:30); Reverse primer—GGAAGACGAAGAGGTTTCTC (SEQ ID NO:31).

Quantitative real time PCR compared the relative levels of mRNA from the two Mallada signata silk genes, MalXBFibroin and MalXBsFib, in the pool of total RNA isolated for cDNA library construction. An Applied Biosystems 7000 sequencing detection machine with iTaq plus ROX master mix (BioRad) was used according to manufacturer's instructions. Relative levels of gene expression were determined by comparing a threshold cycle for amplification for the four primer pairs at different library concentrations ( 1/1000- 1/100,000 dilutions) within the same experiment.

Expression of the MalXBFibroin and MalXBsFib genes in individual insects was assessed by reverse-transcriptase PCR using a Superscript™III One-Step RT-PCR System with Platinum® Taq High Fidelity (Invitrogen; Carlsbad, Calif.) and 12 ng template RNA. PCR reaction conditions were 55° C. for 30 sec, 94° C. for 2 min followed by 35 cycles of 94° C. for 15 sec, 60° C. for 30 sec, 68° C. for 15 sec and a final incubation at 68° C. for 5 min. RNA quality was verified by conducting parallel reactions using the elongation factor primer sets.

Reverse-transcription PCR experiments demonstrated that both MalXBFibroin and MalXBsFib are expressed in adult female Mallada signata but not in larvae or adult males (data not shown). This is the expected expression pattern for silk genes associated with egg laying. Duplicate quantitative real-time PCR experiments measured the comparative abundance of MalXBFibroin and MalXBsFib mRNAs in adult female colleterial silk glands. The ratio found was 7.7±3.5:1 respectively. This is consistent with the 7:1 ratio observed in the cDNA library clones.

### Example 6

### Analysis of Mallada signata Egg Stalk Silk for Malxbfibroin and MalXBsFib

Lacewing egg stalks were analysed by liquid chromatography followed by tandem mass spectrometry as described in Sutherland et al. (2006). Briefly, egg stalks were placed in a zipplate well (Millipore) and digested in sequencing grade trypsin (Promega), with resultant peptides bound to C18 material, washed and eluted. The peptide solution was separated by an Agilent Zorbax SB-C18 5 μm 150×0.5 mm liquid chromatography column then ionized by an electrospray ion source fitted with a micro-nebuliser and analysed on an Agilent XCT ion trap mass spectrometer. Silk proteins were identified using Agilent's Spectrum Mill software to match the peptide mass spectral data with predicted protein sequences from the colleterial silk gland cDNA library.

Mallada signata egg stalk silk was digested with trypsin and analyzed by liquid chromatography mass spectroscopy. The mass spectral data from the tryptic peptides was matched to the predicted proteins encoded by the clones from the silk gland cDNA library. Spectrum Mill software (Agilent) confidently identified the MalXBsFib protein as present in the egg stalk silk (three peptide matches). MalXBFibroin could not be identified in the silk by this method as its protein sequence is not amenable to digestion by trypsin (or any other common mass spectroscopy enzyme). However given that MalXBFibroin is far more abundant in the silk gland than MalXBsFib, once MalXBsFib is identified in the silk it seems likely that both proteins are present.

The inventors had an insufficient quantity of Mallada signata egg stalk silk for accurate amino acid analysis. However the amino acid composition of green lacewing egg stalk silk from the species Chrysopa flava was previously reported (Lucas et al., 1957). The predicted amino acid composition of a 7:1 molar ratio of MalXBFibroin to MalXBsFib is remarkably close to the measured amino acid composition of Chrysopa flava egg stalk (FIG. 1). This data suggests firstly that the protein makeup of Mallada signata and Chrysopa flava egg stalk silks are similar, and secondly that MalXBFibroin and MalXBsFib are the major protein constituents of Mallada signata egg stalk silk.

### Example 7

### The Lacewing Silk Proteins can be Readily Expressed in Recombinant Systems

The inventors identified several features of both the cross beta silk genes and proteins that suggest that these genes are more amenable to recombinant expression than the silks of spiders and silkworm. These features were not predicted by earlier work on this silk.

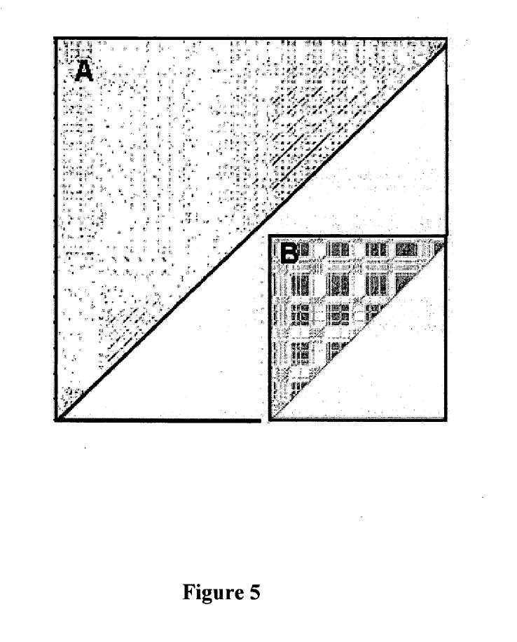

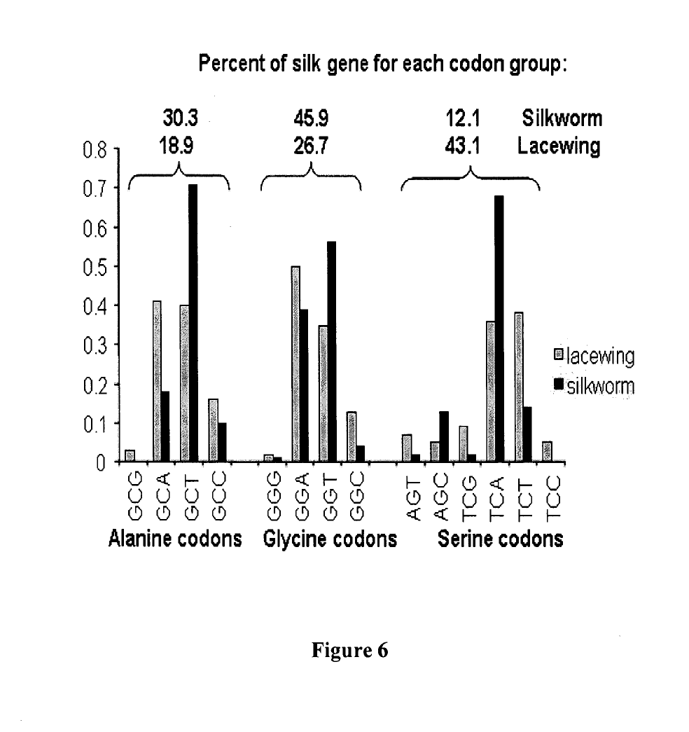

The features of the lacewing genes that make them more amenable to recombinant expression than the silk of Lepidoptera (silkworm) include that the genes are small (<3500 bp) with a less repetitive structure than that observed in silkworm (MalXBFibroin compared with silkworm in FIG. 5) and with a less biased codon usage than that observed in silkworm (MalXBFibroin compared with silkworm in FIG. 6).

The PCR products of MalXBFibroin and MalXBsFib are inserted into pGEMTEasy (Promega) and transformed into Escherichia coli JM109 Competent cells (Stratagene). Correct MalXBFibroin and MalXBsFib inserts in pGEMTEasy are checked by sequencing the vector isolated from the JM109 clone using the QIAGEN plasmid miniprep kit (QIAGEN). The correct insert is then excised by restriction digest, ligated into pET14b (Promega), and transformed into JM109. Correct MalXBFibroin and MalXBsFib inserts in pET14b are again checked by sequencing the vectors isolated from the JM109 clones using the QIAGEN plasmid miniprep kit, and pET14b containing the correct insert is then transformed into E. coli Rosetta™ 2(DE3) Singles™.

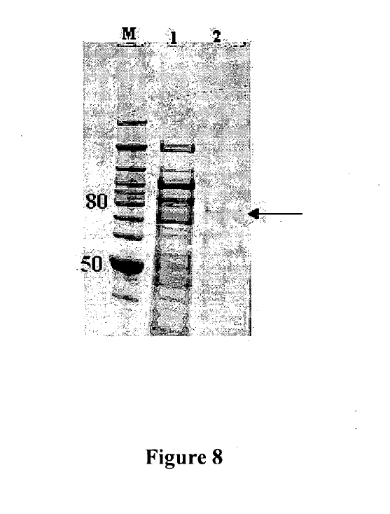

Cells were grown in 5 mL Overnight Express Instant TB Medium (Novagen Cat # 71491-3) containing Overnight Express™ Autoinduction System 1 (Novagen Cat # 71300-3), 100 μg/mL ampicillin and 34 μg/mL chloramphenicol. Cells were grown at 37° C. for 4 hours and growth was continued at 32° C. for 30 hours. Cells were harvested by centrifugation at 13200 rpm in desktop centrifuge for 5 mins and lysed with 150 μL of detergent Bug Buster (Novagen Master Mix Cat# 71456) and incubated on shaker for 1 hour before centrifuging at high speed for 20 mins. To the supernatant equal volume of sterile milliQ® water was added and treated at 70° C. for 30 mins. Sample was centrifuged at high speed for 10 mins. Aliquots (10 μL) of pellet and supernatant with 10 μL of NuPAGE® LDS Sample Buffer (4×) (Invitrogen Cat# NP0007) and 1 μL NuPAGE® Sample Reducing Agent (10×) (Invitrogen Cat# NP0004) was applied to NuPAGE® Novex 4-12% Bis-Tris, (Invitrogen Cat# NP0322BOX) run in NuPAGE® MES SDS Running Buffer-20× (Invitrogen Cat # NP0002) in presence of NuPAGE® Antioxidant (Invitrogen Cat # NP0005). The lacewing silk gene expression is evident in the supernatant at 80 KDa (FIG. 8).

### Example 8

### The Lacewing Silk Proteins are Readily Drawn into Threads

Recombinant proteins are solubilised in various reagents known to solubilise silk proteins including salts such as lithium bromide, detergents such as SDS or denaturing agents such as guanidinium. The denaturant/salt/detergent is removed by dialysis and the protein solution concentrated using commercially available reagents (such as ‘Slide-A-Lyzer Concentrating Solution’ a protein concentrating reagents available from Piece). A drop of the concentrated silk protein solution is deposited onto a substrate and then silk fibres can be drawn from this.

### Example 9

### Expression of Lacewing Silk Proteins in Transgenic Plants

A plant expression vector encoding a silk protein of the invention may consist of a recombinant nucleic acid molecule coding for said protein (for example a polynucleotide provided in any one of SEQ ID NO's:12 to 22) placed downstream of the CaMV 35S promoter in a binary vector backbone containing a kanamycin-resistance gene (NptII).

For the polynucleotides comprising any one of SEQ ID NO's 12 to 22 the construct further may comprise a signal peptide encoding region such as Arabidopsis thaliana vacuolar basic chitinase signal peptide, which is placed in-frame and upstream of the sequence encoding the silk protein.

The construct carrying a silk protein encoding polypeptide is transformed separately into Agrobacterium tumefaciens by electroporation prior to transformation into Arabidopsis thaliana. The hypocotyl method of transformation can be used to transform A. thaliana which can be selected for survival on selective media comprising kanamycin media. After roots are formed on the regenerates they are transferred to soil to establish primary transgenic plants.

Verification of the transformation process can be achieved via PCR screening. Incorporation and expression of polynucleotide can be measured using PCR, Southern blot analysis and/or LC/MS of trypsin-digested expressed proteins.

Two or more different silk protein encoding constructs can be provided in the same vector, or numerous different vectors can be transformed into the plant each encoding a different protein.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

The present application claims priority from U.S. 60/954,189, the entire contents of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Bendtsen et al. (2004) J. Mol. Biol. 340:783-795.
Bini et al. (2004) J. Mol. Biol. 335:27-40.
Cookson et al. (2006) J. Synchrotron Rad. 13: 440-444.
Craig and Riekel (2002) Comp Biochem. and Phys. Part B 133:247-255.
Donald and Windle (1992) Liquid Crystalline Polymers Cambridge University Press, Cambridge.
Duelli (1984) Oviposition. In: The biology of Chrysopidae, eds M. Canard, Y.
Semeria and T. R. New, Dr W. Junk Publishers: The Hague, pp 129-134.
Fuchs and Alix (2005) Proteins: Struct. Funct. Bioinf. 59:828-839.
Geddes et al. (1968) J. Mol. Biol. 32:343-358.
Green et al. (1983) EMBO J. 2:1357-1365.
Harayama (1998) Trends Biotech. 16:76-82.
Harpaz et al. (1994) Structure 2:641-649.
Hepurn et al. (1979) Insect Biochem. 9:66.
Iizuka (1965) Biorheology 3:1-8.
Kenchington (1983) J. Insect Physiol. 29:355-361.
Lucas et al. (1957) Nature 179:609-907.
Needleman and Wunsch (1970) J. Mol. Biol., 48, 443-453.
Parker and Rudall (1957) Nature 179:73-96.
Reiser et al. (1992) Nucleic Acids Research 32 (Web Server issue): W321-W326.
Rudall (1962) In Comparative Biochemistry (ed: Florkin and Mason) 4:297-435 Academic Press, New York.
Rudall and Kenchington (1971) Annual review of Entomology 16:73-96.
Sutherland (2006) Genome Res. 16:1414-1421.

1. An isolated and/or exogenous polynucleotide which encodes a silk polypeptide, wherein at least a portion of silk comprising the polypeptide has a cross beta structure.

2. The polynucleotide of claim 1, wherein the polynucleotide comprises:
i) a sequence of nucleotides as provided in any one of SEQ ID NO's 12 to 20;
ii) a sequence of nucleotides encoding a polypeptide comprising an amino acid sequence as provided in any one of SEQ ID NO's 1 to 9;
iii) a sequence of nucleotides encoding a polypeptide comprising an amino acid sequence which is at least 30% identical to any one or more of SEQ ID NO's 1 to 9;
iv) a sequence of nucleotides encoding a biologically active fragment of ii) or iii);
v) a sequence of nucleotides which is at least 30% identical to any one or more of SEQ ID NO's 12 to 20;
vi) a sequence which hybridizes to any one of i) to v) under stringent conditions,
vii) a sequence of nucleotides as provided in SEQ ID NO:21 or SEQ ID NO:22;
viii) a sequence of nucleotides encoding a polypeptide comprising an amino acid sequence as provided in SEQ ID NO:10 or SEQ ID NO:11;
ix) a sequence of nucleotides encoding a polypeptide comprising an amino acid sequence which is at least 30% identical to SEQ ID NO:10 and/or SEQ ID NO:11;
x) a sequence of nucleotides encoding a biologically active fragment of ii) or iii);
xi) a sequence of nucleotides which is at least 30% identical to SEQ ID NO:21 and/or SEQ ID NO:22; and/or
xii) a sequence which hybridizes to any one of i) to v) under stringent conditions.

3. (canceled)

4. A vector comprising at least one polynucleotide of claim 1.

5. (canceled)

6. A host cell comprising at least one exogenous polynucleotide of claim 1.

7. (canceled)

8. A substantially purified and/or recombinant silk polypeptide, wherein at least a portion of silk comprising the polypeptide has a cross beta structure.

9. The polypeptide of claim 8 which comprises at least 30% serine, at least 15% glycine and at least 15% alanine, and/or which comprises a beta sheet comprising at least 50 strands, wherein each strand is 8 amino acids in length.

10. (canceled)

11. The polypeptide of claim 8 which comprises:
i) an amino acid sequence as provided in any one of SEQ ID NO's 1 to 9;
ii) an amino acid sequence which is at least 30% identical to any one or more of SEQ ID NO's 1 to 9;
iii) a biologically active fragment of i) or ii);
iv) an amino acid sequence as provided in SEQ ID NO:10 or SEQ ID NO:11;
v) an amino acid sequence which is at least 30% identical to SEQ ID NO:10 and/or SEQ ID NO:11; and/or
vi) a biologically active fragment of iv) or v).

12. The polypeptide of parts i) to iii) of claim 11 which comprises between 38% and 48% serine, between 22% and 32% glycine, and between 14% and 24% alanine, or parts iv) to vi) of claim 11 which comprises between 29% and 39% serine, between 16% and 26% glycine, and between 21% and 31% alanine.

13-16. (canceled)

17. The polypeptide of claim 8 which is fused to at least one other polypeptide.

18. A transgenic plant or non-human animal comprising an exogenous polynucleotide of claim 1, the polynucleotide encoding at least one silk polypeptide wherein at least a portion of silk comprising the polypeptide has a cross beta structure.

19. (canceled)

20. A process for preparing a polypeptide of claim 8, the process comprising cultivating a host cell of claim 6 under conditions which allow expression of the polynucleotide encoding the polypeptide, and recovering the expressed polypeptide.

21. An isolated and/or recombinant antibody which specifically binds a polypeptide of claim 8.

22. A silk fiber comprising at least one polypeptide of claim 8.

23. A copolymer comprising at least two polypeptides of claim 8.

24. (canceled)

25. A product comprising at least one polypeptide of claim 8.

26. (canceled)

27. A composition comprising at least one polypeptide of claim 8, and one or more acceptable carriers.

28-29. (canceled)

30. A composition comprising at least one polynucleotide of claim 1, and one or more acceptable carriers.

31. A method of treating or preventing a disease, the method comprising administering a composition comprising at least one drug for treating or preventing the disease and a pharmaceutically acceptable carrier, wherein the pharmaceutically acceptable carrier comprises at least one polypeptide of claim 8.

32-34. (canceled)

35. A product comprising at least one silk fiber of claim 22.

36. A product comprising at least one copolymer of claim 23.

37. A composition comprising at least one silk fiber of claim 22, and one or more acceptable carriers.

38. A composition comprising at least one copolymer of claim 23, and one or more acceptable carriers.

39. A method of treating or preventing a disease, the method comprising administering a composition comprising at least one drug for treating or preventing the disease and a pharmaceutically acceptable carrier, wherein the pharmaceutically acceptable carrier comprises at least one silk fiber of claim 22.

40. A method of treating or preventing a disease, the method comprising administering a composition comprising at least one drug for treating or preventing the disease and a pharmaceutically acceptable carrier, wherein the pharmaceutically acceptable carrier comprises at least one copolymer of claim 23.
